(19) United States
(12) Patent Application Publication
(10) Pub. No.: US 2024/0101569 A1
(43) Pub. Date: Mar. 28, 2024

(54) SALT CRYSTALS

(71) Applicant: INTRA-CELLULAR THERAPIES, INC., New York, NY (US)

(72) Inventor: Peng LI, New Milford, NJ (US)

(21) Appl. No.: 18/263,302

(22) PCT Filed: Jan. 27, 2022
(86) PCT No.: PCT/US2022/070368

§ 371 Date: Jul. 27, 2023

Related U.S. Application Data
(60) Provisional application No. 63/142,073, filed on Jan. 27, 2021.

(51) Int. Cl. C07D 487/14

(52) U.S. Cl. CPC C07D 487/14

(57) ABSTRACT

The present disclosure relates to free base and salt crystals of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one, composition comprising the same and the method of making and using such free base and salt crystals.

### FIELD OF THE DISCLOSURE

The present disclosure relates to acid addition salts and salt crystals of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one, composition comprising the same and the method of making and using such salts and salt crystals.

### BACKGROUND OF THE DISCLOSURE

The compound 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one is disclosed in WO 2014/151409. This compound has been found to be a potent and selective phosphodiesterase 1 (PDE 1) inhibitor useful for the treatment or prophylaxis of disorders characterized by low levels of cAMP and/or cGMP in cells expressing PDE1, neurodegenerative disorders, mental disorders, circulatory and cardiovascular disorders, respiratory and inflammatory disorders, diseases that may be alleviated by the enhancement of progesterone-signalling such as female sexual dysfunction, traumatic brain injury, or any disease or condition characterized by reduced dopamine D1 receptor-signalling activity. This list of disorders is exemplary and not intended to be exhaustive.

The publication WO 2014/151409 discloses 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one, but no specific salt was shown to have particular stability or desired properties. Because many pharmaceutical compounds can exist in different physical forms (e.g., liquid or solid in different crystalline, amorphous, polymorphous, hydrate or solvate forms) which can vary the stability, solubility, bioavailability or pharmacokinetics (absorption, distribution, metabolism, excretion or the like) and/or bioequivalency of a drug, it is of critical importance in the pharmaceutical development to identify a pharmaceutical compound of optimal physical form (e.g., free base or salt in solid, liquid, crystalline, hydrate, solvate, amorphous or polymorphous forms).

### SUMMARY OF THE DISCLOSURE

In a first aspect, the present disclosure is directed to compound 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one free base (“Compound A”) in crystalline form [Free Base Crystal 1]. These free base crystals are stable and are especially advantageous in the preparation of the salt crystals of said Compound A, e.g., succinate, adipate and/or citrate salt crystals. Therefore, in the first aspect, the disclosure provides the following:
1.1 Free Base Crystal 1, wherein the free base crystal is in non-solvate form.
1.2 Any of the preceding Free Base Crystals, wherein the free base crystal is in solvate form.
1.3 Any of the preceding Free Base Crystals, wherein the free base crystal is in solvate form with alcohol.
1.4 Any of the preceding Free Base Crystals, wherein the free base crystal is in solvate form with methanol, ethanol, propanol (e.g., n-propanol or isopropanol) or butanol (e.g., n-butanol).
1.5 Any of the preceding Free Base Crystals, wherein the free base crystal is in non-hydrate or hydrate form.
1.6 Any of the preceding Free Base Crystals, wherein the Free Base Crystals exhibit an X-ray powder diffraction pattern comprising at least five peaks having 2-theta angle values selected from the group consisting of: 9.3, 14.0, 14.7, 17.3, 17.9, 18.7, 21.2, 23.2, 23.3, and 23.7 degrees, wherein the XRPD pattern is measured in a diffractometer using copper anode, e.g., at wavelength alpha1 of 1.5406 Å and wavelength alpha2 of 1.5444 Å.
1.7 Any of the preceding Free Base Crystals, wherein the Free Base Crystals exhibit an X-ray powder diffraction pattern comprising peaks having 2-theta angle values selected from the group consisting of: 9.3, 14.0, 23.2, 23.3, and 23.7 degrees, wherein the XRPD pattern is measured in a diffractometer using copper anode, e.g., at wavelength alpha1 of 1.5406 Å and wavelength alpha2 of 1.5444 Å.
1.8 Any of the preceding Free Base Crystals, wherein the Free Base crystals exhibit an X-ray powder diffraction pattern comprising at least five peaks having 2-theta angle values selected from those set forth in Table 1 below:

TABLE 1

| Index | Angle | d Value | Relative Intensity |
|---|---|---|---|
| 1 | 8.463 | 10.43942 | 10.30% |
| 2 | 9.273 | 9.52959 | 100.00% |
| 3 | 10.513 | 8.40796 | 15.70% |
| 4 | 11.127 | 7.94511 | 8.80% |
| 5 | 13.976 | 6.33155 | 50.60% |
| 6 | 14.682 | 6.02876 | 44.70% |
| 7 | 15.597 | 5.67686 | 2.60% |
| 8 | 16.013 | 5.53037 | 1.70% |
| 9 | 17.326 | 5.11397 | 16.00% |
| 10 | 17.92 | 4.94603 | 22.40% |
| 11 | 18.693 | 4.74307 | 23.90% |
| 12 | 19.667 | 4.5103 | 10.60% |
| 13 | 20.676 | 4.29236 | 7.80% |
| 14 | 21.19 | 4.18944 | 28.90% |
| 15 | 21.668 | 4.09807 | 4.10% |
| 16 | 23.224 | 3.82688 | 60.80% |
| 17 | 23.253 | 3.82216 | 60.40% |
| 18 | 23.461 | 3.78877 | 82.40% |
| 19 | 24.46 | 3.63624 | 7.70% |
| 20 | 25.237 | 3.52604 | 2.00% |
| 21 | 25.849 | 3.4439 | 3.40% |
| 22 | 26.286 | 3.38768 | 2.20% |
| 23 | 27.027 | 3.29646 | 2.80% |
| 24 | 27.536 | 3.23668 | 4.40% |
| 25 | 28.109 | 3.17193 | 6.80% |
| 26 | 28.27 | 3.15429 | 7.20% |
| 27 | 28.934 | 3.08335 | 2.90% |
| 28 | 29.61 | 3.01454 | 3.10% |
| 29 | 30.859 | 2.89533 | 1.70% |
| 30 | 31.311 | 2.85448 | 1.60% |
| 31 | 32.24 | 2.77439 | 2.60% |
| 32 | 33.974 | 2.6366 | 1.50% |
| 33 | 34.98 | 2.56308 | 1.20% |
| 34 | 42.743 | 2.11379 | 2.60% |
| 35 | 43.882 | 2.06156 | 1.10% |

wherein the XRPD pattern is measured in a diffractometer using copper anode, at wavelength alpha1 of 1.5406 Å and wavelength alpha2 of 1.5444 Å.
1.9 Any of the preceding Free Base Crystals, wherein said Free Base Crystals exhibit an X-ray powder diffraction pattern comprising at least five peaks having d-spacing values selected from the group consisting of 9.53, 6.33, 6.02, 5.11, 4.95, 4.74, 4.19, 3.83, 3.82, 3.79 Å.
1.10 Any of the preceding Free Base Crystals, wherein said Free Base Crystals exhibit an X-ray powder diffraction pattern comprising peaks having d-spacing values selected from the group consisting of 9.53, 6.33, 3.83, 3.82, 3.79 Å.
1.11 Any of the preceding Free Base Crystals, wherein said free base crystal exhibits an X-ray powder diffraction pattern corresponding with or substantially as depicted in FIG. 1.
1.12 Any of the preceding Free Base Crystals, wherein said free base crystal exhibits a Differential Scanning Calorimetry (DSC) pattern comprising an endothermic peak at about 195° C.-196° C.
1.13 Any of the preceding Free Base Crystals, wherein the crystal exhibits a Differential Scanning Calorimetry (DSC) pattern corresponding with or substantially as depicted in FIG. 2.
1.14 Any of the preceding Free Base Crystals, wherein said crystal exhibits a Thermogravimetric Analysis (TGA) pattern corresponding with or substantially as depicted in FIG. 3.
1.15 Any of the preceding Free Base Crystals, wherein the crystal has a platelet shape.

In further aspects, the present disclosure is directed to crystals of stable acid addition salts of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one (“Compound A”), e.g., crystallinic acid addition salts with particular acids. These salt crystals are especially advantageous in the preparation of galenic formulations of various and diverse kind. Therefore, in the first aspect, the present disclosure provides the following:
1.1 Compound A in an acid addition salt form, e.g., selected from the group consisting of citrate, adipate, tartrate (e.g., L-tartrate), malate, succinate, gluconate (e.g., D-gluconate), maleate, fumarate, aspartate (e.g., L-aspartate), hippurate, sebacate, glycolate, galactarate, benzoate, pamoate, oxalate and malonate.
1.2 The salt according to formula 1.1, wherein the salt is a succinate salt.
1.3 The salt according to formula 1.1 or 1.2, wherein the salt is a succinate salt having a free base to succinic acid molar ratio of 1:1 (i.e., mono-succinate salt) or 2:1.
1.4 The salt according to formula 1.1, wherein the salt is a citrate salt.
1.5 The salt according to formula 1.1, wherein the salt is an adipate salt.
1.6 The salt according to formula 1.1, wherein the salt is a tartrate (e.g., L-tartrate) salt.
1.7 The salt according to formula 1.1, wherein the salt is a malate (e.g., L-malate) salt.
1.8 The salt according to formula 1.1, wherein the salt is a gluconate (e.g., D-gluconate) salt.

The salt according to any of formulae 1.1-1.8 is referred herein as the Salt(s) of the present disclosure.

It has also been surprisingly found that particular Salts of the present disclosure are in crystalline form, and therefore are preferred for galenic and/or therapeutic use. Therefore, in further embodiments, the present disclosure provides the following:
1.9 The Salt according to any of formulas 1.1-1.8, in crystalline form (hereinafter “Salt Crystals”).
1.10 The salt crystal according to formula 1.9, wherein the salt is a succinate salt.
1.11 The salt crystal according to formula 1.9-1.10, wherein the salt is a succinate salt having a free base to succinic acid molar ratio of 1:1 (i.e., mono-succinate salt) or 2:1.
1.12 The Salt Crystals according to formula 1.9-1.11, wherein the salt is a mono-succinate salt.
1.13 The Salt Crystals according to formula 1.9-1.12, wherein said salt crystals have a plate-like morphology.
1.14 The Salt Crystals according to any of formulae 1.9-1.13, wherein the Salt Crystals exhibit an X-ray powder diffraction pattern comprising at least five peaks having 2-theta angle values selected from the group consisting of: 7.8, 8.2, 11.6, 14.5, 16.5, 18.6, 19.7, 20.4, 20.6, 22.1, 23.3, 24.8, 26.0, and 28.5 degrees, wherein the XRPD pattern is measured in a diffractometer using copper anode, e.g., at wavelength alpha1 of 1.5406 Å and wavelength alpha2 of 1.5444 Å.
1.15 The Salt Crystals according to any of formulae 1.9-1.14, wherein the Salt Crystals exhibit an X-ray powder diffraction pattern comprising peaks having 2-theta angle values selected from the group consisting of: 7.8, 8.2, 11.6, 16.5, and 20.4 degrees, wherein the XRPD pattern is measured in a diffractometer using copper anode, e.g., at wavelength alpha1 of 1.5406 Å and wavelength alpha2 of 1.5444 Å.
1.16 The Salt Crystals according to any of formulae 1.9-1.15, wherein the salt crystals exhibit an X-ray powder diffraction pattern comprising at least five peaks having 2-theta angle values selected from those set forth in Table 2 below:

TABLE 2

| Index | Angle | d Value | Relative Intensity |
|---|---|---|---|
| 1 | 4.637 | 19.04004 | 2.70% |
| 2 | 6.731 | 13.12075 | 0.70% |
| 3 | 7.769 | 11.37029 | 26.70% |
| 4 | 8.207 | 10.76495 | 100.00% |
| 5 | 8.477 | 10.42187 | 3.10% |
| 6 | 8.744 | 10.10468 | 0.90% |
| 7 | 9.318 | 9.48322 | 0.70% |
| 8 | 9.811 | 9.00791 | 8.90% |
| 9 | 10.564 | 8.36782 | 1.20% |
| 10 | 11.599 | 7.62307 | 26.20% |
| 11 | 12.022 | 7.35594 | 7.50% |
| 12 | 13.436 | 6.58468 | 3.00% |
| 13 | 14.542 | 6.08639 | 10.00% |
| 14 | 15.633 | 5.66376 | 1.10% |
| 15 | 16.459 | 5.38151 | 59.40% |
| 16 | 17.233 | 5.1416 | 4.90% |
| 17 | 17.287 | 5.12569 | 4.50% |
| 18 | 17.926 | 4.94427 | 0.70% |
| 19 | 18.589 | 4.76952 | 15.80% |
| 20 | 19.141 | 4.63309 | 7.30% |
| 21 | 19.695 | 4.50388 | 14.70% |
| 22 | 20.368 | 4.35656 | 37.00% |
| 23 | 20.589 | 4.31035 | 17.00% |
| 24 | 21.207 | 4.18612 | 2.70% |
| 25 | 22.085 | 4.02163 | 16.40% |
| 26 | 22.354 | 3.97397 | 1.30% |
| 27 | 22.827 | 3.89265 | 2.00% |
| 28 | 23.331 | 3.80965 | 14.60% |
| 29 | 23.944 | 3.71355 | 0.60% |
| 30 | 24.426 | 3.64128 | 2.70% |
| 31 | 24.791 | 3.58851 | 24.30% |
| 32 | 25.254 | 3.52377 | 1.40% |
| 33 | 25.961 | 3.42934 | 14.10% |
| 34 | 27.063 | 3.29211 | 4.30% |
| 35 | 27.478 | 3.24341 | 3.00% |
| 36 | 28.056 | 3.17783 | 1.10% |
| 37 | 28.461 | 3.13356 | 13.10% |
| 38 | 28.723 | 3.10556 | 6.90% |
| 39 | 29.804 | 2.99535 | 4.30% |
| 40 | 30.106 | 2.96597 | 6.70% |
| 41 | 30.511 | 2.92749 | 0.80% |
| 42 | 31.053 | 2.87764 | 2.00% |
| 43 | 31.718 | 2.81883 | 3.10% |
| 44 | 32.867 | 2.72281 | 1.10% |
| 45 | 33.208 | 2.69565 | 4.10% |
| 46 | 33.849 | 2.6461 | 1.00% |
| 47 | 34.671 | 2.58522 | 2.30% |
| 48 | 35.609 | 2.51919 | 0.50% |
| 49 | 36.687 | 2.44764 | 3.00% |
| 50 | 37.432 | 2.40058 | 0.60% |
| 51 | 38.516 | 2.33547 | 0.90% |
| 52 | 39.735 | 2.26661 | 0.80% |
| 53 | 40.89 | 2.20522 | 0.50% |
| 54 | 41.762 | 2.16117 | 0.50% |
| 55 | 42.536 | 2.12359 | 0.50% |
| 56 | 43.691 | 2.0701 | 0.90% |
| 57 | 44.609 | 2.02964 | 0.60% |

wherein the XRPD pattern is measured in a diffractometer using copper anode, at wavelength alpha1 of 1.5406 Å and wavelength alpha2 of 1.5444 Å.
1.17 The Salt Crystals according to any of formulae 1.9-1.16, wherein said salt crystals exhibit an X-ray powder diffraction pattern comprising at least five peaks having d-spacing values selected from the group consisting of 11.37, 10.77, 7.62, 6.09, 5.38, 4.77, 4.50, 4.36, 4.31, 4.02, 3.81, 3.59, 3.43, and 3.13 Å.
1.18 The Salt Crystals according to any of formulae 1.9-1.17, wherein said salt crystals exhibit an X-ray powder diffraction pattern comprising peaks having d-spacing values selected from the group consisting of 11.37, 10.77, 7.62, 5.38, and 4.36 Å.
1.19 The Salt Crystals according to any of formulae 1.9-1.18, wherein said salt crystals exhibit an X-ray powder diffraction pattern comprising at least five peaks having d-spacing values selected from those set forth in Table 2 of formula 1.16.
1.20 The Salt Crystals according to any of formulae 1.9-1.19, wherein said salt crystals exhibit an X-ray powder diffraction pattern corresponding with or substantially as set forth in Table 2 of formula 1.16.
1.21 The Salt Crystals according to any of formulae 1.9-1.20, wherein said salt crystals exhibit an X-ray powder diffraction pattern corresponding with or substantially as depicted in FIG. 4.
1.22 The Salt Crystals according to any of formulae 1.9-1.21, wherein said salt crystal exhibits a Differential Scanning Calorimetry (DSC) pattern comprising an endothermic peak at about 177° C.-178° C.
1.23 The Salt Crystals according to any of formulae 1.9-1.22, wherein said salt crystal exhibits a Differential Scanning Calorimetry (DSC) pattern corresponding with or substantially as depicted in FIG. 5.
1.24 The Salt Crystals according to any of formulae 1.9-1.23, wherein said salt crystals exhibit a Thermogravimetric Analysis (TGA) pattern corresponding with or substantially as depicted in FIG. 6.
1.25 The Salt Crystals according to any of formulae 1.9-1.24, wherein said salt crystals are prepared by reacting 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one free base crystals in ethanol with succinic acid.
1.26 The salt crystal according to formula 1.9, wherein the salt is a citrate salt.
1.27 The Salt Crystals according to formula 1.26, wherein the salt is a mono-citrate salt.
1.28 The Salt Crystals according to any of formulae 1.26-1.27, wherein the Salt Crystals exhibit an X-ray powder diffraction pattern comprising at least five peaks having 2-theta angle values selected from the group consisting of: 5.9, 7.0, 7.8, 8.8, 11.7, 11.9, 13.2, 13.8, 14.4, 15.7, 16.1, 16.3, 16.8, 18.1, 19.0, 19.9, 20.2, 20.7, 21.0, 21.3, 22.4, 23.6, 24.9, 25.3, and 27.2 degrees, wherein the XRPD pattern is measured in a diffractometer using copper anode, e.g., at wavelength alpha1 of 1.5406 Å and wavelength alpha2 of 1.5444 Å.
1.29 The Salt Crystals according to any of formulae 1.26-1.28, wherein the Salt Crystals exhibit an X-ray powder diffraction pattern comprising at least five peaks having 2-theta angle values selected from the group consisting of: 5.9, 7.0, 7.8, 8.8, 11.7, 11.9, 14.4, 15.6, 16.1, 16.8, 18.1, 19.0, 21.0, and 24.9 degrees, wherein the XRPD pattern is measured in a diffractometer using copper anode, e.g., at wavelength alpha1 of 1.5406 Å and wavelength alpha2 of 1.5444 Å.
1.30 The Salt Crystals according to any of formulae 1.26-1.29, wherein the Salt Crystals exhibit an X-ray powder diffraction pattern comprising peaks having 2-theta angle values selected from the group consisting of: 5.9, 7.0, 8.8, 16.1, and 16.8 degrees, wherein the XRPD pattern is measured in a diffractometer using copper anode, e.g., at wavelength alpha1 of 1.5406 Å and wavelength alpha2 of 1.5444 Å.
1.31 The Salt Crystals according to any of formulae 1.26-1.30, wherein the salt crystals exhibit an X-ray powder diffraction pattern comprising at least five peaks having 2-theta angle values selected from those set forth in Table 3 below:

TABLE 3

| Index | Angle | d Value | Relative Intensity |
|---|---|---|---|
| 1 | 5.899 | 14.96978 | 4.70% |
| 2 | 6.973 | 12.66677 | 100.00% |
| 3 | 7.796 | 11.3313 | 40.00% |
| 4 | 8.085 | 10.92637 | 4.00% |
| 5 | 8.766 | 10.07923 | 75.00% |
| 6 | 9.734 | 9.07874 | 2.80% |
| 7 | 10.088 | 8.76119 | 2.90% |
| 8 | 11.657 | 7.58558 | 32.90% |
| 9 | 11.931 | 7.41146 | 29.90% |
| 10 | 12.868 | 6.87432 | 3.40% |
| 11 | 13.156 | 6.72442 | 14.80% |
| 12 | 13.808 | 6.40803 | 19.70% |
| 13 | 13.97 | 6.33415 | 9.40% |
| 14 | 14.423 | 6.13647 | 28.60% |
| 15 | 15.061 | 5.87759 | 7.60% |
| 16 | 15.617 | 5.66965 | 22.30% |
| 17 | 16.147 | 5.48477 | 61.40% |
| 18 | 16.34 | 5.42028 | 12.50% |
| 19 | 16.812 | 5.26929 | 70.70% |
| 20 | 17.824 | 4.97236 | 8.90% |
| 21 | 18.085 | 4.90126 | 37.80% |
| 22 | 18.976 | 4.6729 | 32.00% |
| 23 | 19.305 | 4.59406 | 8.10% |
| 24 | 19.84 | 4.47137 | 10.70% |
| 25 | 20.187 | 4.3953 | 14.40% |
| 26 | 20.712 | 4.28513 | 13.30% |
| 27 | 21.011 | 4.2248 | 57.70% |
| 28 | 21.254 | 4.17707 | 12.50% |
| 29 | 22.065 | 4.02531 | 9.80% |
| 30 | 22.382 | 3.96901 | 13.40% |
| 31 | 23.619 | 3.76377 | 11.10% |
| 32 | 24.235 | 3.66947 | 3.80% |
| 33 | 24.949 | 3.5661 | 20.50% |
| 34 | 25.328 | 3.51362 | 11.00% |
| 35 | 26.484 | 3.36286 | 7.10% |
| 36 | 27.008 | 3.29876 | 9.80% |
| 37 | 27.222 | 3.27326 | 11.50% |
| 38 | 27.663 | 3.22208 | 4.10% |
| 39 | 29.532 | 3.02227 | 5.70% |
| 40 | 30.101 | 2.96647 | 4.70% |
| 41 | 30.83 | 2.89796 | 9.30% |
| 42 | 32.38 | 2.76265 | 4.60% |
| 43 | 38.313 | 2.34741 | 4.60% |

wherein the XRPD pattern is measured in a diffractometer using copper anode, at wavelength alpha1 of 1.5406 Å and wavelength alpha2 of 1.5444 Å.
1.32 The Salt Crystals according to any of formulae 1.26-1.31, wherein said salt crystals exhibit an X-ray powder diffraction pattern comprising at least five peaks having d-spacing values selected from the group consisting of 14.97, 12.67, 11.33, 10.08, 7.59, 7.41, 6.72, 6.41, 6.14, 5.67, 5.48, 5.42, 5.27, 4.90, 4.67, 4.47, 4.39, 4.29, 4.22, 4.18, 3.97, 3.76, 3.57, 3.51, and 3.27 Å.
1.33 The Salt Crystals according to any of formulae 1.26-1.31, wherein said salt crystals exhibit an X-ray powder diffraction pattern comprising at least five peaks having d-spacing values selected from the group consisting of 14.97, 12.67, 11.33, 10.08, 7.59, 7.41, 6.14, 5.67, 5.48, 5.27, 4.90, 4.67, 4.22, and 3.57 Å.
1.34 The Salt Crystals according to any of formulae 1.26-1.31, wherein said salt crystals exhibit an X-ray powder diffraction pattern comprising peaks having d-spacing values selected from the group consisting of 14.97, 12.67, 10.08, 5.48, and 5.27 Å.
1.35 The Salt Crystals according to any of formulae 1.26-1.34, wherein said salt crystals exhibit an X-ray powder diffraction pattern comprising at least five peaks having d-spacing values selected from those set forth in Table 3 of formula 1.31.
1.36 The Salt Crystals according to any of formulae 1.26-1.35, wherein said salt crystals exhibit an X-ray powder diffraction pattern corresponding with or substantially as set forth in Table 3 of formula 1.31.
1.37 The Salt Crystals according to any of formulae 1.26-1.36, wherein said salt crystals exhibit an X-ray powder diffraction pattern corresponding with or substantially as depicted in FIG. 7.
1.38 The Salt Crystals according to any of formulae 1.26-1.37, wherein said salt crystal exhibits a Differential Scanning Calorimetry (DSC) pattern comprising an endothermic peak at about 142° C.-144° C.
1.39 The Salt Crystals according to any of formulae 1.26-1.37, wherein said salt crystal exhibits a Differential Scanning Calorimetry (DSC) pattern corresponding with or substantially as depicted in FIG. 8.
1.40 The Salt Crystals according to any of formulae 1.26-1.39, wherein said salt crystals exhibit a Thermogravimetric Analysis (TGA) pattern corresponding with or substantially as depicted in FIG. 9.
1.41 The Salt Crystals according to any of formulae 1.26-1.40, wherein said salt crystals are prepared by reacting 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one free base crystals in acetone with citric acid.
1.42 The salt crystal according to formula 1.9, wherein the salt is an adipate salt.
1.43 The Salt Crystals according to any of formulae 1.42, wherein the Salt Crystals exhibit an X-ray powder diffraction pattern comprising at least five peaks having 2-theta angle values selected from the group consisting of: 5.4, 6.4, 7.1, 9.6, 10.9, 14.2, 15.5, 15.7, 16.1, 16.5, 17.9, 20.8, 21.8, 22.4, 23.9, 24.7, 26.3, and 27.8 degrees, wherein the XRPD pattern is measured in a diffractometer using copper anode, e.g., at wavelength alpha1 of 1.5406 Å and wavelength alpha2 of 1.5444 Å.
1.44 The Salt Crystals according to any of formulae 1.42-1.43, wherein the Salt Crystals exhibit an X-ray powder diffraction pattern comprising at least five peaks having 2-theta angle values selected from the group consisting of: 5.4, 6.4, 7.1, 9.6, 10.9, 16.1, 16.45, 17.9, 23.9, and 24.7 degrees, wherein the XRPD pattern is measured in a diffractometer using copper anode, e.g., at wavelength alpha1 of 1.5406 Å and wavelength alpha2 of 1.5444 Å.
1.45 The Salt Crystals according to any of formulae 1.42-1.44, wherein the Salt Crystals exhibit an X-ray powder diffraction pattern comprising peaks having 2-theta angle values selected from the group consisting of: 5.4, 6.4, 9.6, 16.5, and 24.7 degrees, wherein the XRPD pattern is measured in a diffractometer using copper anode, e.g., at wavelength alpha1 of 1.5406 Å and wavelength alpha2 of 1.5444 Å.
1.46 The Salt Crystals according to any of formulae 1.42-1.45, wherein the salt crystals exhibit an X-ray powder diffraction pattern comprising at least five peaks having 2-theta angle values selected from those set forth in Table 4 below:

TABLE 4

| Index | Angle | d Value | Relative Intensity |
|---|---|---|---|
| 1 | 5.439 | 16.23483 | 43.00% |
| 2 | 6.438 | 13.71747 | 95.60% |
| 3 | 7.07 | 12.49342 | 27.90% |
| 4 | 9.628 | 9.17907 | 100.00% |
| 5 | 10.918 | 8.09738 | 40.30% |
| 6 | 12.394 | 7.13586 | 1.40% |
| 7 | 12.923 | 6.84504 | 1.80% |
| 8 | 14.195 | 6.23423 | 11.40% |
| 9 | 15.525 | 5.70302 | 16.40% |
| 10 | 15.684 | 5.6455 | 13.90% |
| 11 | 16.098 | 5.50148 | 40.20% |
| 12 | 16.45 | 5.38442 | 57.00% |
| 13 | 16.712 | 5.30055 | 4.90% |
| 14 | 17.596 | 5.0362 | 4.70% |
| 15 | 17.937 | 4.94129 | 32.10% |
| 16 | 18.683 | 4.74569 | 6.60% |
| 17 | 19.498 | 4.54904 | 5.40% |
| 18 | 19.615 | 4.52223 | 5.90% |
| 19 | 19.939 | 4.4494 | 8.90% |
| 20 | 20.833 | 4.26041 | 23.20% |
| 21 | 21.362 | 4.1562 | 5.50% |
| 22 | 21.756 | 4.08176 | 12.40% |
| 23 | 22.443 | 3.95834 | 23.50% |
| 24 | 23.307 | 3.81343 | 5.90% |
| 25 | 23.913 | 3.71817 | 29.30% |
| 26 | 24.694 | 3.60233 | 44.10% |
| 27 | 25.6 | 3.47692 | 1.60% |
| 28 | 26.324 | 3.38294 | 11.20% |
| 29 | 26.609 | 3.34724 | 4.40% |
| 30 | 27.451 | 3.24647 | 2.60% |
| 31 | 27.761 | 3.21095 | 10.90% |
| 32 | 28.26 | 3.15538 | 5.50% |
| 33 | 28.899 | 3.08708 | 3.10% |
| 34 | 29.329 | 3.04273 | 5.30% |
| 35 | 30.595 | 2.91963 | 4.60% |
| 36 | 31.481 | 2.83947 | 1.20% |
| 37 | 32.091 | 2.78691 | 4.00% |
| 38 | 32.598 | 2.74467 | 2.00% |
| 39 | 33.046 | 2.70847 | 1.70% |
| 40 | 34.479 | 2.59915 | 1.50% |
| 41 | 34.818 | 2.57461 | 1.90% |
| 42 | 35.455 | 2.5298 | 1.00% |
| 43 | 36.353 | 2.46936 | 2.10% |
| 44 | 37.01 | 2.42703 | 1.50% |

wherein the XRPD pattern is measured in a diffractometer using copper anode, at wavelength alpha1 of 1.5406 Å and wavelength alpha2 of 1.5444 Å.
1.47 The Salt Crystals according to any of formulae 1.42-1.46, wherein said salt crystals exhibit an X-ray powder diffraction pattern comprising at least five peaks having d-spacing values selected from the group consisting of 16.23, 13.72, 12.49, 9.18, 8.10, 6.23, 5.70, 5.65, 5.50, 5.38, 4.94, 4.26, 4.08, 3.96, 3.72, 3.60, 3.38, and 3.21 Å.
1.48 The Salt Crystals according to any of formulae 1.42-1.46, wherein said salt crystals exhibit an X-ray powder diffraction pattern comprising at least five peaks having d-spacing values selected from the group consisting of 16.23, 13.72, 12.49, 9.18, 8.10, 5.50, 5.38, 4.94, 3.72, and 3.60 Å.
1.49 The Salt Crystals according to any of formulae 1.42-1.46, wherein said salt crystals exhibit an X-ray powder diffraction pattern comprising peaks having d-spacing values selected from the group consisting of 16.23, 13.72, 9.18, 5.38, and 3.60 Å.
1.50 The Salt Crystals according to any of formulae 1.42-1.49, wherein said salt crystals exhibit an X-ray powder diffraction pattern comprising at least five peaks having d-spacing values selected from those set forth in Table 4 of formula 1.46.
1.51 The Salt Crystals according to any of formulae 1.42-1.50, wherein said salt crystals exhibit an X-ray powder diffraction pattern corresponding with or substantially as set forth in Table 4 of formula 1.46.
1.52 The Salt Crystals according to any of formulae 1.42-1.51, wherein said salt crystals exhibit an X-ray powder diffraction pattern corresponding with or substantially as depicted in FIG. 10.
1.53 The Salt Crystals according to any of formulae 1.42-1.52, wherein said salt crystal exhibits a Differential Scanning Calorimetry (DSC) pattern comprising an endothermic peak at about 170° C.-172° C.
1.54 The Salt Crystals according to any of formulae 1.42-1.53, wherein said salt crystals exhibit a Thermogravimetric Analysis (TGA) and a Differential Scanning Calorimetry (DSC) pattern corresponding with or substantially as depicted in FIG. 11.
1.55 The Salt Crystals according to any of formulae 1.42-1.54, wherein said salt crystals are prepared by reacting 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one free base crystals in a solvent (e.g., ethanol, acetone, or ethyl acetate) with adipic acid.
1.56 The Salt Crystal according to formula 1.9, wherein the salt is a malate salt.
1.57 The Salt Crystal according to formula 1.56, wherein the salt is an L-malate salt.
1.58 The Salt Crystals according to any of formulae 1.56-1.57, wherein said salt crystals exhibit an X-ray powder diffraction pattern corresponding with or substantially as depicted in FIG. 12.
1.59 The Salt Crystals according to any of formulae 1.56-1.58, wherein said salt crystal exhibits a Differential Scanning Calorimetry (DSC) pattern comprising an endothermic peak at about 214° C.-215° C.
1.60 The Salt Crystals according to any of formulae 1.56-1.59, wherein said salt crystals exhibit a Thermogravimetric Analysis (TGA) and a Differential Scanning Calorimetry (DSC) pattern corresponding with or substantially as depicted in FIG. 13.
1.61 The Salt Crystal according to formula 1.9, wherein the salt is a tartrate salt.
1.62 The Salt Crystal according to formula 1.61, wherein the salt is an L-tartrate salt.
1.63 The Salt Crystals according to any of formulae 1.61-1.62, wherein said salt crystals exhibit an X-ray powder diffraction pattern corresponding with or substantially as depicted in FIG. 14.
1.64 The Salt Crystals according to any of formulae 1.61-1.63, wherein said salt crystal exhibits a Differential Scanning Calorimetry (DSC) pattern comprising an endothermic peak at about 240° C.-242° C.
1.65 The Salt Crystals according to any of formulae 1.61-1.64, wherein said salt crystals exhibit a Thermogravimetric Analysis (TGA) and a Differential Scanning Calorimetry (DSC) pattern corresponding with or substantially as depicted in FIG. 15.
1.66 The Salt Crystal according to formula 1.9, wherein the salt is a gluconate salt.
1.67 The Salt Crystal according to formula 1.66, wherein the salt is a D-gluconate salt.
1.68 The Salt Crystals according to any of formulae 1.66-1.67, wherein said salt crystals exhibit an X-ray powder diffraction pattern corresponding with or substantially as depicted in FIG. 16.
1.69 The Salt Crystals according to any of formulae 1.66-1.68, wherein said salt crystal exhibits a Differential Scanning Calorimetry (DSC) pattern comprising an endothermic peak at about 195° C.-196° C.
1.70 The Salt Crystals according to any of formulae 1.66-1.69, wherein said salt crystals exhibit a Thermogravimetric Analysis (TGA) and a Differential Scanning Calorimetry (DSC) pattern corresponding with or substantially as depicted in FIG. 17.
1.71 Salt Crystals according to any of the above formulae, wherein said Salt Crystals are in a single crystal form and are free or substantially free of any other form, e.g., less than 10 wt. %, preferably less than about 5 wt. %, more preferably less than about 2 wt. %, still preferably less than about 1 wt. %, still preferably less than about 0.1%, most preferably less than about 0.01 wt. % of amorphous form.
1.72 Salt Crystals according to any of the above formulae, wherein said Salt Crystals are in a single crystal form and are free or substantially free of any other form, e.g., less than 10 wt. %, preferably less than about 5 wt. %, more preferably less than about 2 wt. %, still preferably less than about 1 wt. %, still preferably less than about 0.1%, most preferably less than about 0.01 wt. % of other crystal forms.
1.73 Salt Crystals according to any of the above formulae, wherein said Salt Crystals are in a single crystal form and are free or substantially free of any other form, e.g., less than 10 wt. %, preferably less than about 5 wt. %, more preferably less than about 2 wt. %, still preferably less than about 1 wt. %, still preferably less than about 0.1%, most preferably less than about 0.01 wt. % of amorphous and other crystal forms.
1.74 Salt Crystals according to any of the preceding formulae when made by any of processes described or similarly described in any of Methods 1, et seq. or any of Examples 1-6.

In a further aspect, the present disclosure also provides a process [Method 1] for the production of stable acid addition salts of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one (“Compound A”), e.g., crystallinic acid addition salts with particular acids, comprising the steps of reacting Compound A in free base form with an acid in a solvent and isolating the salt obtained. In particular embodiments, the present disclosure provides the following:
1.1 Method 1, wherein the acid is selected from citric acid, adipic acid, tartaric acid (e.g., L-tartaric acid), malic acid, succinic acid, gluconic acid (e.g., D-gluconic acid), maleic acid, fumaric acid, aspartic acid (e.g., L-aspartic acid), hippuric acid, sebacic acid, glycolic acid, galactaric acid, benzoic acid, pamoic acid, oxalic acid and malonic acid.
1.2 Any of the preceding Methods, wherein the solvent is an alcohol (e.g., methanol and/or ethanol), acetone, acetonitrile, dimethyl sulfoxide (DMSO), ethyl acetate, and/or toluene.
1.3 Any of the preceding Methods, wherein 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one (Compound A) is in crystalline form.
1.4 Method 1.3, wherein Compound A is in non-solvate form.
1.5 Any of the Methods 1.1-1.3, wherein Compound A is in solvate form.
1.6 Methods 1.5, wherein Compound A is in solvate form with alcohol (solvate form with methanol, ethanol, propanol (e.g., n-propanol or isopropanol) or butanol (e.g., n-butanol)).
1.7 Any of the Methods 1.3-1.6, wherein Compound A is in solvate form with methanol, ethanol, propanol (e.g., n-propanol or isopropanol) or butanol (e.g., n-butanol).
1.8 Any of the Methods 1.3-1.7, wherein Compound A is in non-hydrate or hydrate form.
1.9 Any of the Methods 1.3-1.8, wherein Compound A exhibits an X-ray powder diffraction pattern corresponding with or substantially as depicted in FIG. 1.
1.10 Any of the Methods 1.3-1.9, wherein Compound A exhibits a Differential Scanning Calorimetry (DSC) pattern comprising an endothermic peak at about 195° C.-196° C.
1.11 Any of the Methods 1.3-1.10, wherein Compound A exhibits a Differential Scanning Calorimetry (DSC) pattern corresponding with or substantially as depicted in FIG. 2.
1.12 Any of the Methods 1.3-1.11, wherein Compound A has a platelet shape.
1.13 Any of the preceding Methods, wherein the acid is in the amount of about 2 molar equivalents relative to Compound A.
1.14 Any of Methods 1.1-1.12, wherein the acid is in the amount of about 1 molar equivalent relative to Compound A.
1.15 Any of Methods 1.1-1.12, wherein the acid is in the amount of about 0.5 molar equivalent relative to Compound A.
1.16 Any of the preceding Methods, wherein the acid is in aqueous, hydrate or crystalline form.
1.17 Any of the preceding Methods, wherein the acid is succinic acid.
1.18 Method 1.17, wherein the solvent is an alcohol.
1.19 Any of Methods 1.17-1.18, wherein the solvent is ethanol.
1.20 Any of Methods 1.17-1.19, wherein Compound A is dissolved in ethanol.
1.21 Any of Methods 1.17-1.20, wherein the solution of Compound A in ethanol is further heated to an elevated temperature (e.g., to a temperature of about 65° C. to about 70° C., e.g., about 67° C., e.g., until all solids are dissolved).
1.22 Any of Methods 1.17-1.21, wherein the succinic acid is dissolved in the ethanol.
1.23 Any of Methods 1.17-1.18, further comprising the step of heating the mixture of Compound A and the acid in the solvent to about 75° C. to about 80° C. (e.g., about 78° C.).
1.24 Any of Methods 1.1-1.16, wherein the acid is citric acid.
1.25 Method 1.24, wherein the solvent is acetone.
1.26 Any of the preceding Methods, further comprising the optional step of seeding the reaction mixture.
1.27 Any of the preceding Methods, wherein the reaction mixture/solution is optionally sonicated.
1.28 Any of the preceding Methods, further comprising the step of isolating the crystals thus obtained.
1.29 Any of the preceding Methods, further comprising the step of drying the crystals thus obtained (e.g., in an oven at about 45° C., by vacuum or combinations thereof).
1.30 Any of Methods 1.1-1.16, wherein the acid is adipic acid.
1.31 Method 1.30, wherein the solvent is ethanol, acetone, or ethyl acetate.
1.32 Method 1.30 or 1.31, wherein the resulting solution is heated to about 50° C.

A method [Method 2] for the prophylaxis or treatment of a patient, e.g., a human suffering from a disorder selected from the following disorders:
A. Neurodegenerative diseases, including Parkinson's disease, restless leg, tremors, dyskinesias, Huntington's disease, Alzheimer's disease, and drug-induced movement disorders;
B. Mental disorders, including depression, attention deficit disorder, attention deficit hyperactivity disorder, bipolar illness, anxiety, sleep disorders, e.g., narcolepsy, cognitive impairment, e.g., cognitive impairment of schizophrenia, dementia, Tourette's syndrome, autism, fragile X syndrome, psychostimulant withdrawal, and drug addiction;
C. Circulatory and cardiovascular disorders, including cerebrovascular disease, stroke, congestive heart disease, hypertension, pulmonary hypertension, e.g., pulmonary arterial hypertension, and sexual dysfunction, including cardiovascular diseases and related disorders as described in International Application No. PCT/US2014/16741, the contents of which are incorporated herein by reference;
D. Respiratory and inflammatory disorders, including asthma, chronic obstructive pulmonary disease, and allergic rhinitis, as well as autoimmune and inflammatory diseases;
E. Diseases that may be alleviated by the enhancement of progesterone-signaling such as female sexual dysfunction;
F. A disease or disorder such as psychosis, glaucoma, or elevated intraocular pressure;
G. Traumatic brain injury;
H. Cancers or tumors, e.g., brain tumors, a glioma (e.g., ependymoma, astrocytoma, oligodendrogliomas, brain stem glioma, optic nerve glioma, or mixed gliomas, e.g., oligoastrocytomas), an astrocytoma (e.g., glioblastoma multiforme), osteosarcoma, melanoma, leukemia, neuroblastoma or leukemia;
I. Renal disorders, e.g., kidney fibrosis, chronic kidney disease, renal failure, glomerulosclerosis and nephritis;
J. Any disease or condition characterized by low levels of cAMP and/or cGMP (or inhibition of cAMP and/or cGMP signaling pathways) in cells expressing PDE1; and/or
K. Any disease or condition characterized by reduced dopamine D1 receptor signaling activity,
   comprising administering to a patient in need thereof a therapeutically effective amount of (a) the compound 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one in acid addition salt form according to any of Free Base Crystal 1 et seq., or the Salt Crystal 1 et seq. of the present disclosure.
2.1 A pharmaceutical composition comprising any of Free Base Crystal 1 et seq., or the Salt Crystal 1 et seq. for use as a medicament, e.g., for use in the manufacture of a medicament for the treatment or prophylaxis of a disease as described in Method 2.

### BRIEF DESCRIPTION OF THE DRAWINGS

FIG. 1 depicts an x-ray powder diffraction pattern of the free base crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

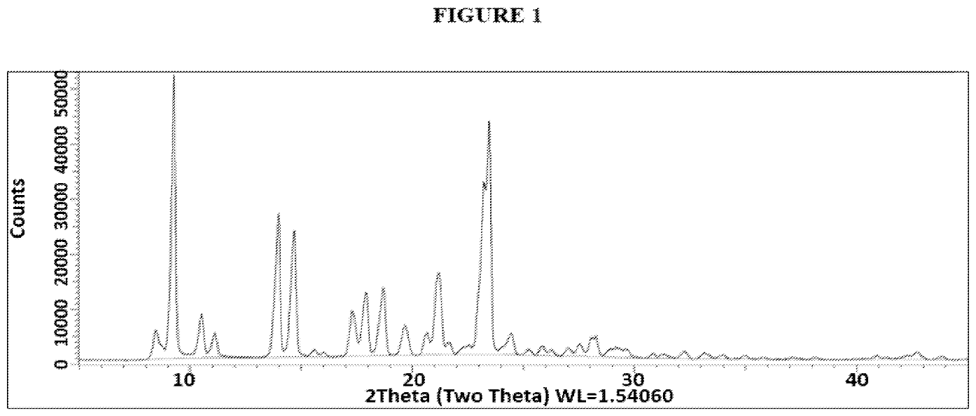

FIG. 2 depicts a differential scanning calorimetry (DSC) thermograph of the free base crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

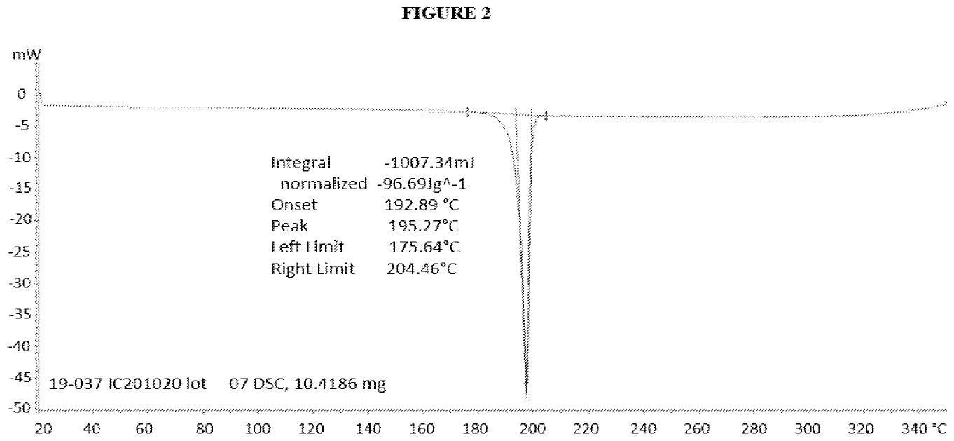

FIG. 3 depicts a thermogravimetric analysis (TGA) thermograph of the free base crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

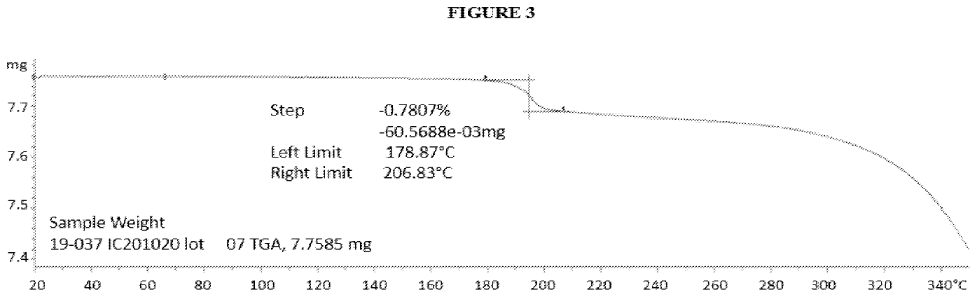

FIG. 4 depicts an x-ray powder diffraction pattern of the succinate salt crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

FIG. 5 depicts a differential scanning calorimetry (DSC) thermograph pattern of the succinate salt crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

FIG. 6 depicts a thermogravimetric analysis (TGA) thermograph pattern of the succinate salt crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

FIG. 7 depicts an x-ray powder diffraction pattern of the citrate salt crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

FIG. 8 depicts a differential scanning calorimetry (DSC) thermograph of the citrate salt crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

FIG. 9 depicts a thermogravimetric analysis (TGA) thermograph of the citrate salt crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

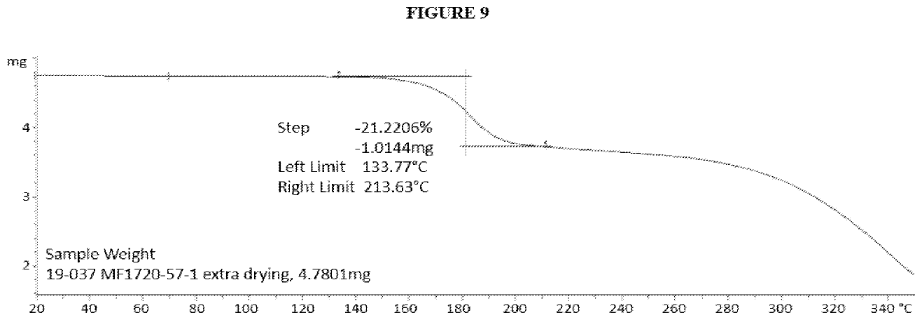

FIG. 10 depicts an x-ray powder diffraction pattern of the adipate salt crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

FIG. 11 depicts a thermogravimetric analysis (TGA) and differential scanning calorimetry (DSC) thermograph pattern of the adipate salt crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

FIG. 12 depicts an x-ray powder diffraction pattern of the malate salt crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

FIG. 13 depicts a thermogravimetric analysis (TGA) and differential scanning calorimetry (DSC) thermograph pattern of the malate salt crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

FIG. 14 depicts an x-ray powder diffraction pattern of the tartrate salt crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

FIG. 15 depicts a thermogravimetric analysis (TGA) and differential scanning calorimetry (DSC) thermograph pattern of the tartrate salt crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

FIG. 16 depicts an x-ray powder diffraction pattern of the gluconate salt crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

FIG. 17 depicts a thermogravimetric analysis (TGA) and differential scanning calorimetry (DSC) thermograph pattern of the gluconate salt crystal of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one.

### DETAIL DESCRIPTION

As use herein, the term “crystal” or “crystals” or “crystalline” or “crystallinic” refers to any solid that has a short or long range order of the molecules, atoms or ions in a fixed lattice arrangement. Salt Crystals of the Present Disclosure may be in a single crystal form. Therefore, the Salt Crystals of the Present Disclosure may be in a triclinic, monoclinic, orthorhombic, tetragonal, rhobohedral, hexagonal or cubic crystal form or mixtures thereof. In particular, the Salt Crystals of the Present Disclosure are in dry crystalline form. In another embodiment, the Salt Crystals of the Present Disclosure are in needle form. In still another embodiment, the Salt Crystals of the Present Disclosure are in plate-like form. In a particular embodiment, the Salt Crystals of the Present Disclosure are substantially free of other forms, e.g., free of amorphous or other crystal forms.

The term “substantially free” of other crystal forms refer to less than about 10 wt. %, preferably less than about 5 wt. %, more preferably less than about 2 wt. %, still preferably less than about 1 wt. %, still preferably less than about 0.1%, most preferably less than about 0.01 wt. % of other forms or other crystal forms, e.g., amorphous or other crystal forms.

The term “predominantly” or “substantially entirely in a single form” refers to less than about 10 wt. %, preferably less than about 5 wt. %, more preferably less than about 2 wt. %, still preferably less than about 1 wt. %, still preferably less than about 0.1%, most preferably less than about 0.01 wt. % of other crystal forms, e.g., amorphous or other crystal forms.

In particular embodiment, the crystals of the disclosure may contain trace amounts of solvent, e.g., in solvate form, or trace amounts of water, e.g., in hydrate form. Preferably, the Salt Crystals of the disclosure are in non-solvate form. Still preferably, the crystals of the disclosure are in non-solvate and non-hydrate form.

The Salt Crystals of the disclosure may have a free base to acid ratio of 1 to 1, 1 to 0.5 or 1 to >1, e.g., 1 to 1.3 or 1 to 2, etc. For example, the succinate salt crystal of the disclosure may comprise 1 molar equivalent of the free base to 1 molar equivalent of the succinic acid. Preferably, the succinate salt crystal of the disclosure comprises 1 molar equivalent of the free base to 1 molar equivalent of the succinic acid wherein the acid is a di-acid, such as fumaric acid or tartaric acid, the ratio of free base to acid may be 1 molar equivalent of free base to 0.5 equivalent of the di-acid, e.g., to form a hemi-fumarate or hemi-tartrate salt.

The term “solvate” refers to crystalline solid adducts containing either stoichiometric or nonstoichiometric amounts of a solvent incorporated within the crystal structure. Therefore, the term “non-solvate” form herein refers to salt crystals that are free or substantially free of solvent molecules within the crystal structures of the disclosure. Similarly, the term “non-hydrate” form herein refers to salt crystals that are free or substantially free of water molecules within the crystal structures of the disclosure.

The term “amorphous” form refers to solids of disordered arrangements of molecules and do not possess a distinguishable crystal lattice.

The crystallinity or the morphology of the crystals of the Present Disclosure may be determined by a number of methods, including, but not limited to single crystal X-ray diffraction, X-ray powder diffraction, polarizing optical microscopy, thermal microscopy, differential scanning calorimetry (DSC), thermogravimetric analysis (TGA), infrared adsorption spectroscopy and Raman spectroscopy. Characterization of solvates or hydrates or lack thereof may also be determined by DSC and/or TGA.

It is to be understood that X-ray powder diffraction pattern or the differential scanning calorimetry pattern of a given sample may vary a little (standard deviation) depending on the instrument used, the time and temperature of the sample when measured and standard experimental errors. Therefore, the temperature or the 2-theta values, d-spacing values, heights and relative intensity of the peaks as set forth herein in the Tables or in the Figures will have an acceptable level of deviation. For example, the values may have an acceptable deviation of e.g., about 20%, 15%, 10%, 5%, 3%, 2% or 1%. In particular embodiment, the 2-theta values or the d-spacing values of the XRPD pattern of the crystals of the current disclosure may have an acceptable deviation of ±0.2 degrees and/or ±0.2 Å. Further, the XRPD pattern of the crystals of the disclosure may be identified by the characteristic peaks as recognized by one skilled in the art. For example, the crystals of the disclosure may be identified by e.g., at least five characteristic peaks, e.g., at least three or at least five peaks, e.g., at least three or at least five 2-theta values and/or at least three or at least five d-spacing values as set forth in the XRPD patterns set forth herein. Therefore, the term “corresponding with or substantially as” set forth in any of the Tables or depicted in any of the Figures refers to any crystals which has an XRPD having the major or characteristic peaks as set forth in the tables/figures.

The term “about” in front of a numerical value refers to the numerical value itself ±20%, ±15%, ±10%, preferably ±5%, preferably ±3%, preferably ±2%, preferably ±1% of that value. When referencing temperature, the term about refers to the temperature value itself ±10° C., preferably ±5° C., preferably ±3° C. of the reference temperature. In another example, when referencing 2-theta angle values, the term “about” refers to the numerical 2-theta angle value itself ±0.2 degrees of the reference 2-theta angle value. In still another example, when referencing d-spacing values, the term “about” refers to the numerical 2-theta angle value itself ±0.2 Å of the reference d-spacing value.

The crystals of the disclosure are selective PDE1 inhibitors. Therefore, the crystals of the disclosure are useful for the treatment of PDE1 related disorders as set forth in e.g., WO 2014/151409, WO 2018/049417, WO 2019/227004, WO 2019/152697, WO 2009/075784, WO 2010/132127, WO 2006/133261 and WO 2011/153129, the contents of each of which are incorporated by reference in their entireties.

The term “patient” includes human and non-human. In one embodiment, the patient is a human. In another embodiment, the patient is a non-human.

### Example 1—Preparation of the Succinate Salt Crystals

The succinate salt crystals of the disclosure may be prepared as described or similarly described herein. In a 20 L round bottom flask with reflux condenser, overhead stirrer and temperature probe, 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one (776.00 g, 1 Eq, 1.6851 mol) was suspended in 7.5 L of absolute ethanol. The mixture was heated to 67° C. (internal), and to the suspension was added succinic acid (200.00 g, 1.0051 Eq, 1.6936 mol). Once added, the suspension started to dissolve. The reaction mixture was heated to 78° C., after 15 min giving a clear orange/red solution. The reaction was filtered hot over P3 filter to remove undissolved particles. The mixture was then seeded, left to cool to room temperature, and allowed to stand for 48 h for crystallisation. The reaction mixture was filtered on P2 filter and rinsed twice with 500 mL of EtOH. The solids were collected and dried in circulation oven at 45° C. to constant weight. Yield: 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one succinate (804.8 g, 82.54%). Recrystallization yielded an additional 110 g of material.

A 3 L round bottom flask was loaded with the 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one succinate obtained from the first crystallization (745.0 g, 1 Eq, 1.288 mol) and the 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one succinate obtained from the recrystallization (110.0 g, 0.1477 Eq, 190.1 mmol). To the flask, ethanol (1.0 L) was added and the slurry was stirred for 1 hour on a rotary evaporator at 65° C. to obtain a homogeneous suspension. The water bath temperature was adjusted to 50° C. and the ethanol was removed under reduced pressure (distilling starts at 220 mbar up to 25 mbar) to dryness. The remaining solids (wet weight: 909.5 g) were transferred into a tray, which was dried in a circulation oven at 45° C. for 5 days. 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one succinate (845.5 g, 1.461 mol, 98.89%) was obtained as an off-white solid.

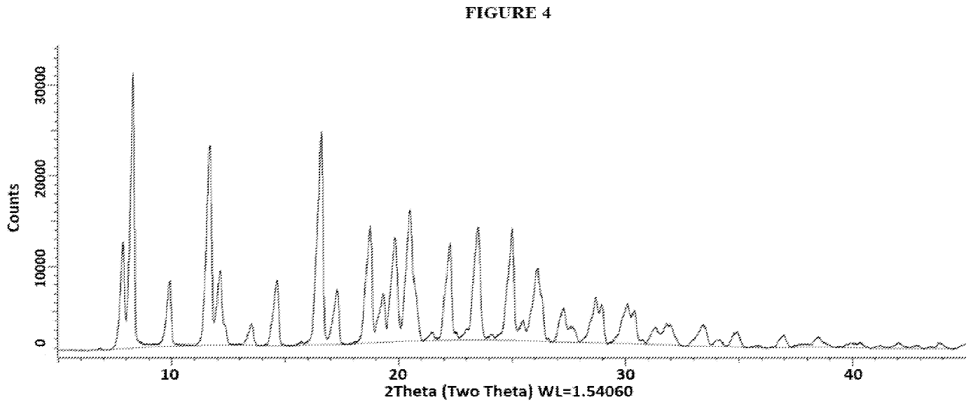

The XRPD of the succinate salt crystals is obtained as described or similarly described herein. The result is depicted in FIG. 4. The X-ray powder diffraction studies are performed using a Bruker AXS D8 discover HTS. Using a Cu anode at 40 kV, 40 mA; Göbel mirror, line optics. Detector: Linear detector LYNXEYE XE with receiving slit 2.95° detector opening. Measurement conditions: scan range 2-45° 2θ, 1 s/step, 0.005°/step, and all measuring conditions are logged in the instrument control file.

The XRPD pattern of the succinate salt crystal is depicted in FIG. 4 and has peaks as set forth below:

| Index | Angle | d Value | Relative Intensity |
|---|---|---|---|
| 1 | 4.637 | 19.04004 | 2.70% |
| 2 | 6.731 | 13.12075 | 0.70% |
| 3 | 7.769 | 11.37029 | 26.70% |
| 4 | 8.207 | 10.76495 | 100.00% |
| 5 | 8.477 | 10.42187 | 3.10% |
| 6 | 8.744 | 10.10468 | 0.90% |
| 7 | 9.318 | 9.48322 | 0.70% |
| 8 | 9.811 | 9.00791 | 8.90% |
| 9 | 10.564 | 8.36782 | 1.20% |
| 10 | 11.599 | 7.62307 | 26.20% |
| 11 | 12.022 | 7.35594 | 7.50% |
| 12 | 13.436 | 6.58468 | 3.00% |
| 13 | 14.542 | 6.08639 | 10.00% |
| 14 | 15.633 | 5.66376 | 1.10% |
| 15 | 16.459 | 5.38151 | 59.40% |
| 16 | 17.233 | 5.1416 | 4.90% |
| 17 | 17.287 | 5.12569 | 4.50% |
| 18 | 17.926 | 4.94427 | 0.70% |
| 19 | 18.589 | 4.76952 | 15.80% |
| 20 | 19.141 | 4.63309 | 7.30% |
| 21 | 19.695 | 4.50388 | 14.70% |
| 22 | 20.368 | 4.35656 | 37.00% |
| 23 | 20.589 | 4.31035 | 17.00% |
| 24 | 21.207 | 4.18612 | 2.70% |
| 25 | 22.085 | 4.02163 | 16.40% |
| 26 | 22.354 | 3.97397 | 1.30% |
| 27 | 22.827 | 3.89265 | 2.00% |
| 28 | 23.331 | 3.80965 | 14.60% |
| 29 | 23.944 | 3.71355 | 0.60% |
| 30 | 24.426 | 3.64128 | 2.70% |
| 31 | 24.791 | 3.58851 | 24.30% |
| 32 | 25.254 | 3.52377 | 1.40% |
| 33 | 25.961 | 3.42934 | 14.10% |
| 34 | 27.063 | 3.29211 | 4.30% |
| 35 | 27.478 | 3.24341 | 3.00% |
| 36 | 28.056 | 3.17783 | 1.10% |
| 37 | 28.461 | 3.13356 | 13.10% |
| 38 | 28.723 | 3.10556 | 6.90% |
| 39 | 29.804 | 2.99535 | 4.30% |
| 40 | 30.106 | 2.96597 | 6.70% |
| 41 | 30.511 | 2.92749 | 0.80% |
| 42 | 31.053 | 2.87764 | 2.00% |
| 43 | 31.718 | 2.81883 | 3.10% |
| 44 | 32.867 | 2.72281 | 1.10% |
| 45 | 33.208 | 2.69565 | 4.10% |
| 46 | 33.849 | 2.6461 | 1.00% |
| 47 | 34.671 | 2.58522 | 2.30% |
| 48 | 35.609 | 2.51919 | 0.50% |
| 49 | 36.687 | 2.44764 | 3.00% |
| 50 | 37.432 | 2.40058 | 0.60% |
| 51 | 38.516 | 2.33547 | 0.90% |
| 52 | 39.735 | 2.26661 | 0.80% |
| 53 | 40.89 | 2.20522 | 0.50% |
| 54 | 41.762 | 2.16117 | 0.50% |
| 55 | 42.536 | 2.12359 | 0.50% |
| 56 | 43.691 | 2.0701 | 0.90% |
| 57 | 44.609 | 2.02964 | 0.60% |

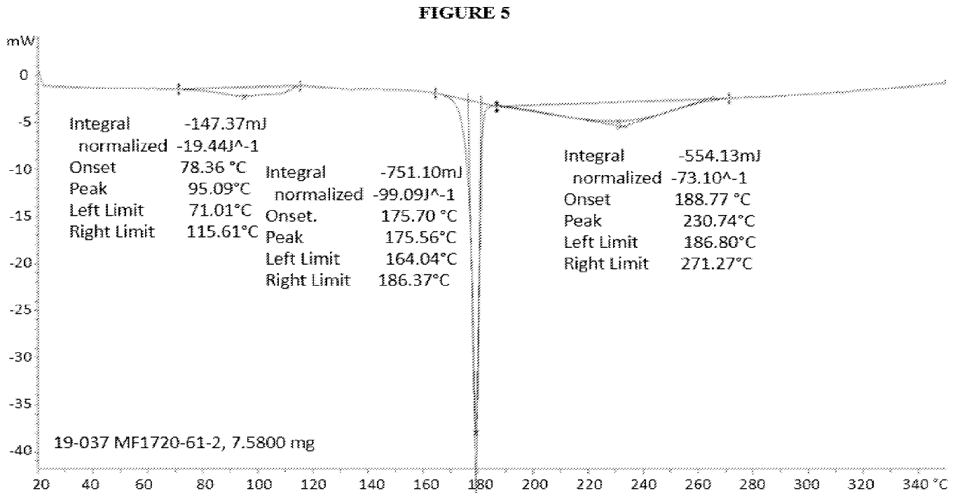

Differential Scanning Calorimetry (DSC) thermograph of the succinate Salt Crystals is obtained as described or similarly described herein and the DSC is depicted in FIG. 5. The DSC studies were performed using a Mettler Toledo DSC1 STARe System. The samples are made using Al crucibles (40 μl; pierced). 1-8 mg of sample is loaded onto a pre-weighed Al crucible and is kept at 20° C. for 5 minutes, after which it is heated at 10° C./min from 20° C. to 350° C. and kept at 350° C. for 1 minute. A nitrogen purge of 40 ml/min is maintained over the sample. The software used for data collection and evaluation is STARe Software v15.00 build 8668. No corrections are applied to the thermogram.

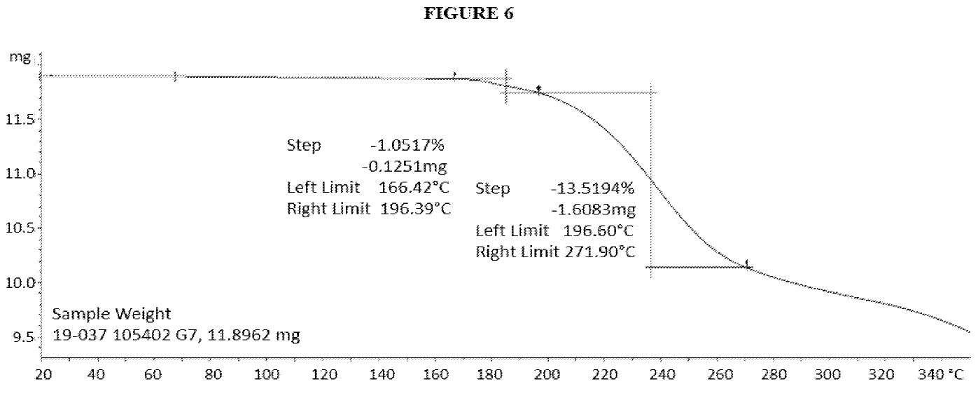

Thermogravimetric Analysis (TGA) & Differential Scanning Calorimetry (DSC) of the succinate salt crystals is obtained as described or similarly described herein and is depicted in FIG. 6. The TGA/DSC studies were performed using a Mettler Toledo TGA/DSC-01/03 STARe System with a 34-position auto sampler. The samples are made using Al crucibles (40 μl; pierced). 5-10 mg of sample is loaded into a pre-weighed Al crucible and is kept at 20° C. for 5 minutes, after which it is heated at 10° C./min from 20° C. to 350° C. A nitrogen purge of 40 ml/min is maintained over the sample. The software used for data collection and evaluation is STARe Software v15.00 build 8668.

The succinate Salt Crystals are particularly stable, has good solubility, low hygroscopicity, a single melting event, definable stoichiometry, has plate-like morphology and are non-solvate, non-hydrate, all of which are desirable properties for galenic formulation.

The method of making the Compound 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one is generally described in WO 2014/151409, the contents of which is incorporated by reference in its entirety. This compound can also be prepared as summarized or similarly summarized in the following reaction scheme.

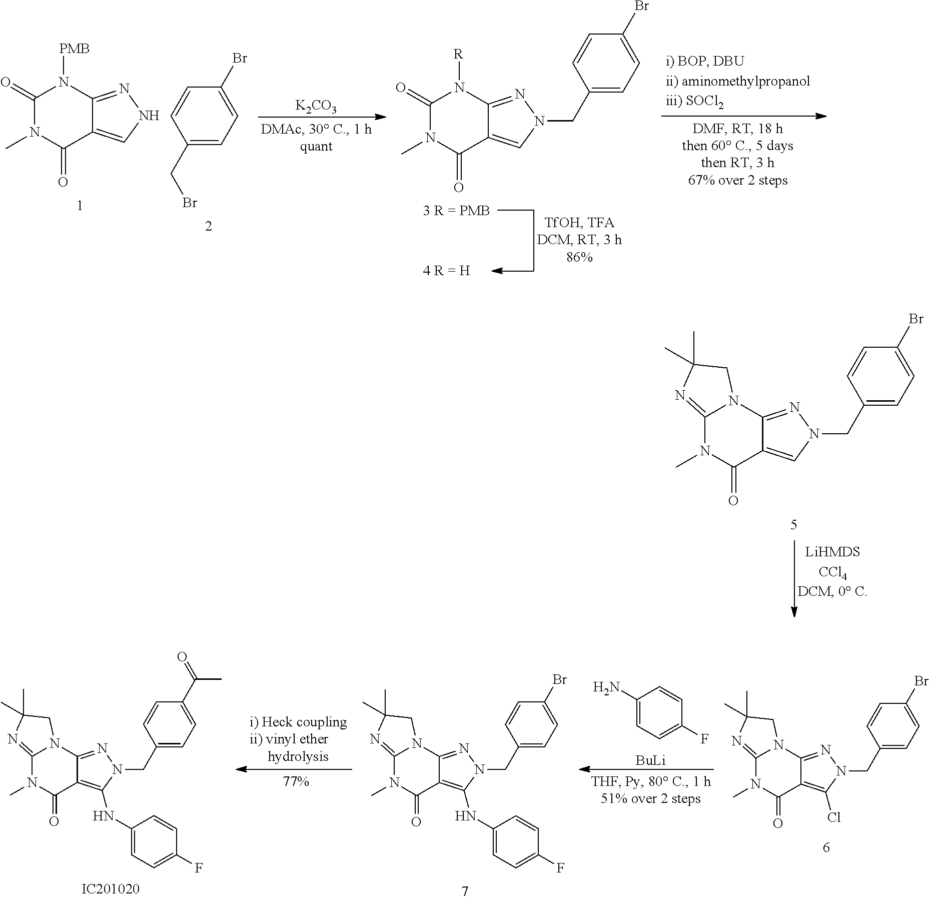

In particular, 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one may be prepared as described or similarly described below.

### Preparation of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one

### 2-(4-bromobenzyl)-7-(4-methoxybenzyl)-5-methyl-2,7-dihydro-4H-pyrazolo[3,4-d]pyrimidine-4,6(5H)-dione (3)

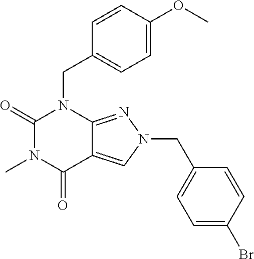 [SMILES: COc1ccc(Cn2c(=O)n(C)c(=O)c3cn(Cc4ccc(Br)cc4)nc32)cc1]

To a clean 70 L reaction, DMAc (10 L) was added, and under stirring (137 rpm) were added 7-(4-methoxybenzyl)-5-methyl-2,7-dihydro-4H-pyrazolo[3,4-d]pyrimidine-4,6(5H)-dione (1477 g, 1 Eq, 5.159 mol), 1-bromo-4-(bromomethyl)benzene (1292 g, 1.002 Eq, 5.169 mol) and potassium carbonate (713.0 g, 1 Eq, 5.159 mol) under nitrogen. DMAc (2.5 L) was added for rinsing the reactor inside. The mixture (suspension) was warmed to 50° C. and stirred at this temperature for 45 minutes. The mixture was warmed to 80° C. and stirred for 30 minutes. IPC (by LC-MS) showed full conversion. The mixture was cooled to 30° C., and a thick white suspension was obtained. The suspension was sucked out of the reactor into a work-up vessel. To the reaction mixture was added water (35 L) at higher stirring (320 rpm). The suspension was filtered off over two large Buchner funnels, washed with water (2×2 L each) and dried in the oven at 45° C. for 20 h. The material was weighed: 3222 g (>100% yield). The batch was split up: 120 g was dried at the small rotavap and dried at 45° C. in the oven overnight. The large batch was dried on the large rotavap and in the oven overnight. Yield small batch: 81.6 g (3%). Yield large batch: 2276 g (96%).

### 2-(4-bromobenzyl)-5-methyl-2,7-dihydro-4H-pyrazolo[3,4-d]pyrimidine-4,6(5H)-dione

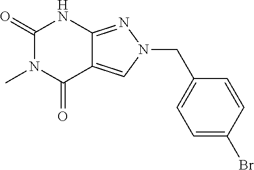 [SMILES: Cn1c(=O)[nH]c2nn(Cc3ccc(Br)cc3)cc2c1=O]

A 20 L reactor vessel was filled with 2-(4-bromobenzyl)-7-(4-methoxybenzyl)-5-methyl-2,7-dihydro-4H-pyrazolo[3,4-d]pyrimidine-4,6(5H)-dione (2276 g, 1 Eq, 4.999 mol). Under mechanical stirring 2,2,2-trifluoroacetic acid (10 kg, 6.7 L, 18 Eq, 88 mol) was added and the mixture was stirred till all was dissolved. T_{end}=25° C. Trifluoromethanesulfonic acid (2251 g, 3.001 Eq, 15.00 mol) was added drop wise. An exothermic effect was noticed. Tₘₐₓ=43.4° C. A purple red solution was obtained. The reaction mixture was left to stir for additional 16 h. The mixture was transferred to the 70 L reactor vessel and cooled to 15° C. Acetonitrile (20 L) was added with a dropping funnel and stirred for 30 minutes, and the red suspension was collected in 10 L tanks. The reactor was filled with a mixture of 28% ammonia in water (21 L) and Acetonitrile (10 L) and cooled to 0° C. The reaction mixture from the 10 L tanks was added in small portions. The mixture (yellow suspension) was stirred for 30 minutes and filtered off over a 8 L P2 glass filter and washed with acetonitrile/water (1:1; 10 L). The yellow solid was stirred in ethyl acetate (10 L) for 1 hour and filtered off and washed with ethyl acetate (3.5 L). The solids were dried at 45° C. Yield: 2-(4-bromobenzyl)-5-methyl-2,7-dihydro-4H-pyrazolo[3,4-d]pyrimidine-4,6(5H)-dione (1437 g, 4.288 mol, 85.79%) white/tan solid.

### 2-(4-bromobenzyl)-5,7,7-trimethyl-7,8-dihydro-2H-imidazo[1,2-a]pyrazolo[4,3-e]pyrimidin-4(5H)-one

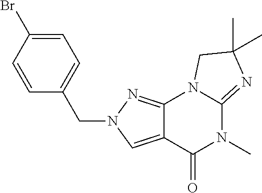 [SMILES: CN1C(=O)c2cn(Cc3ccc(Br)cc3)nc2N2CC(C)(C)N=C12]

A flask of 20 L, equipped with stirrer and temperature probe, was set under nitrogen atmosphere and warmed with a heat gun to get rid of water. DMF (4 L) was added, and under stirring 2-(4-bromobenzyl)-5-methyl-2,7-dihydro-4H-pyrazolo[3,4-d]pyrimidine-4,6(5H)-dione (736.0 g, 1 Eq, 2.196 mol) was added. A suspension was formed. The funnel was rinsed with DMF (200 mL). ((1H-benzo[d][1,2,3]triazol-1-yl)oxy)tris(dimethylamino)phosphonium hexafluorophosphate(V) (1166 g, 1.201 Eq, 2.636 mol) was added and the funnel was rinsed with DMF (200 mL). 2,3,4,6,7,8,9,10-octahydropyrimido[1,2-a]azepine (401.2 g, 1.2 Eq, 2.635 mol) (weighted in a 100 ml beaker and rinsed with DMF (600 mL)) was added. The suspension became a clear brown solution and an exothermic effect was noticed: T₀=19.6° C.; Tₘₐₓ=29.7° C. The mixture was stirred for 16 h. 2-amino-2-methylpropan-1-ol (822.1 g, 4.2 Eq, 9.223 mol), which was molten in the closed bottle in warm water, was added and the mixture, giving a small endothermic effect. The vessel was warmed to 60° C. and stirred for 4 days. The reaction mixture was cooled to 0° C. with ice salt. Add drop wise thionyl chloride (1810 g, 6.929 Eq, 15.22 mol). Temperature was maintained below 20° C. A light brown suspension was formed. Stir after addition for 1 hour.

This step may alternatively be performed as follows. In a 50 L extraction vessel with mechanical stir was added: 24 L ice/water and 7 L 25% ammonia. The reaction mixture was added in portions under stirring (120 rpm). During the addition, ice was added in portions to keep the mixture cold (<15° C.). After addition with Ethyl acetate (1×3 L: sticky solids on bottom extraction vessel; 1× with 10 L, 1×5 L), the organic layers were extracted. The ethyl acetate layers were washed with 0.5 M NaOH solution (2×5 L: first time yellow color after extraction, second time colorless) to remove starting material, 5% NaCl solution (3×5 L, after 2 extractions, the water layer is still light basic), brine (1×5 L; pH is neutral, organic layer clear). The organic layer was dried over Na₂SO₄, filtered and concentrated at the rotavap but not completely: last 1.5 L were not concentrated as a white solid is precipitating. The vessel is cooled in an ice bath and the solids were filtered off and washed with 2×50 mL cold EtOAc. The solids were dried overnight on air. Yield: 566 gram (66%) white solid. Further yield after further evaporation of the filtrate: 19.1 g. 2-(4-bromobenzyl)-3-chloro-5,7,7-trimethyl-7,8-dihydro-2H-imidazo[1,2-a]pyrazolo[4,3-e]pyrimidin-4(5H)-one

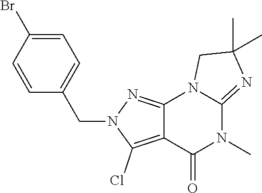 [SMILES: CN1C(=O)c2c(nn(Cc3ccc(Br)cc3)c2Cl)N2CC(C)(C)N=C12]

To the 50 L reactor was added DCM (15 L), followed by 2-(4-bromobenzyl)-5,7,7-trimethyl-7,8-dihydro-2H-imidazo[1,2-a]pyrazolo[4,3-e]pyrimidin-4(5H)-one (1790 g, 1 Eq, 4.610 mol). Added perchloromethane (1.418 kg, 892 mL, 2 Eq, 9.22 mol) and cooled the reaction mixture to −10° C. Added lithium bis(trimethylsilyl)amide (1.466 kg, 8.8 L, 1.9 Eq, 8.76 mol) using a dropping funnel while keeping the temperature between −5° C. and −10° C. Addition is complete after 1 hour and 50 minutes. A sample was checked by HPLC-MS (1 drop reaction mixture in acetonitrile) and showed complete conversion.

The reaction was quenched by addition of saturated aqueous ammonium chloride (15 L). The temperature rose from −6° C. to 5° C. The mixture was stirred for 10 minutes at 5° C. and then warmed to 18° C. The layers were separated. The water layer was extracted with dichloromethane (5 L). The combined organic layers were washed with water (2×5 L). The organic layer was then dried over sodium sulfate and evaporated to dryness. This gave a dark brown/black sticky solid (2520 g). NMR shows desired product combined with toluene and ethyl benzene. Additional solvent was removed by using a high vacuum (oil) pump. This gave a crude yield of 2335 g (120% yield). Overnight the material solidified in the evaporation flask. The material was removed from the flask, powdered and further dried in an open container at room temperature. Compound 6 was obtained as a dark brown solid (2178 g, 5.15 mol, 110%).

### 2-(4-bromobenzyl)-3-((4-fluorophenyl)amino)-5,7,7-trimethyl-7,8-dihydro-2H-imidazo[1,2-a]pyrazolo[4,3-e]pyrimidin-4(5H)-one

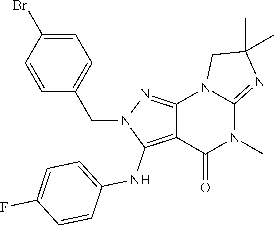 [SMILES: CN1C(=O)c2c(nn(Cc3ccc(Br)cc3)c2Nc2ccc(F)cc2)N2CC(C)(C)N=C12]

To the 50 L reactor under nitrogen atmosphere was added THF (11 L) and 4-fluoroaniline (1.6 kg, 1.3 L, 3.1 Eq, 14 mol). The mixture was cooled to −6° C. Butyllithium (2.5 M in hexane, 0.72 kg, 4.5 L, 2.5 Eq, 11 mol) was added over a period of 70 minutes while keeping the temperature between −5° C. and −2° C. The reaction mixture was warmed to 15° C. over a period of 1 hour. The starting material (Compound 6) (2126 g, 1 Eq, 4.5 mol) was dissolved in pyridine (10 L). This gave a black solution. The solution was added fast to the reaction mixture (within 10 minutes). An exothermic reaction to 29° C. was observed. The reaction mixture was heated at 70° C. for 2 hours. The reaction was cooled to 55° C. (below reflux) and a sample was taken (1 drop reaction mixture in acetonitrile). The reaction mixture was heated at 70° C. for another 2 hours, and stirred overnight. To the reaction mixture was added saturated NH₄Cl (11 L) and the mixture was stirred for 10 minutes. The layers were separated. The water layer (about 22 L) containing solids was extracted with ethyl acetate (3 L). Additional water (3 L) was added and stirred again. This gave 2 clear layers, which were then separated. The water layer (about 16 L) was extracted with ethyl acetate (2 L). The combined organic layer (black) was washed with 5 L half saturated brine. The water layer (7 L) was separated. The organic layer was washed with 3 L half saturated brine. The water layer (3 L) was removed. The organic layer was dried over sodium sulfate, filtered over a glass filter and evaporated to dryness on the large scale rotavap at 50° C. A brown oil was obtained (3346 g).

The crude material was purified over silica gel in four batches. A 20 kg Silica gel column was prepared by pouring as a slurry in dichloromethane. The crude material (3346 g) was dissolved in dichloromethane (1.5 L) to give a 60% stock solution. 1500 g of the stock solution (about 900 g product) was applied on the column. The solution was first eluted with dichloromethane (30 L), and subsequently collected in 10 fractions. Next, it was eluted with dichloromethane/acetone 20% (50 L) and collected in 4.5 L fractions. The fractions were checked by TLC (eluted with DCM/Acetone 20%, colored with PMA dip). The column was then eluted with DCM/acetone 30% (40 L) and collected 2 L fractions. Fraction 14 to 20 were very brown and contain 4-fluoraniline according to the TLC. Finally eluted with DCM/acetone 40% (about 110 L). Obtained 73.7 g starting compound 6 combined with compound 7, 95 g compound 7 with traces of compound 6 and 187.9 g pure product 7.

After the four columns were finished, several batches of similar purities were combined to give a total amount of 803.6 g pure compound 7. The fractions containing above 80% pure compound 7 were combined and stored (303 g).

### 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one

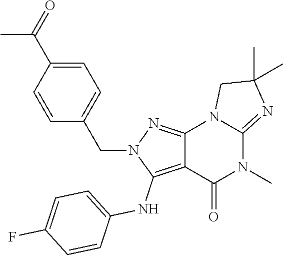 [SMILES: CC(=O)c1ccc(Cn2nc3c(c2Nc2ccc(F)cc2)C(=O)N(C)C2=NC(C)(C)CN23)cc1]

A 20 L reaction vessel equipped with teflon coated metal stirring propeller, reflux condenser and temperature probe was flushed with nitrogen. Prepared 2700 mL of a 1:15 mixture of water and DMF (170 mL demi-water+2530 mL DMF). The reaction vessel was charged with most of the solvent mixture.

Compound 7 was added (800.0 g, 1 Eq, 1.608 mol), followed by 1,3-bis(diphenylphosphaneyl) propane (66.34 g, 0.1 Eq, 160.8 mmol), potassium carbonate (448 g, 2.02 Eq, 3.24 mol) and 1-(vinyloxy)butane (4.833 kg, 6.24 L, 30 Eq, 48.25 mol). The solids were rinsed in with the remaining solvent mixture.

The resulting mixture was degassed by bubbling with nitrogen for 1 h while stirring. Added palladium(II) acetate (18.06 g, 0.05 Eq, 80.42 mmol) and heated to 70° C. over a period of 1 hour. The internal temperature rose to 84° C. (the reaction is probably exothermic!). Allowed to cool back down to 70° C. by lowering the heating mantel. The reaction mixture was stirred at 70° C. overnight.

HPLC analysis showed the conversion was complete. The reaction mixture was cooled to 50° C., then transferred to a 20 L evaporation flask (used 1.5 L water to dissolve the solids) and concentrated until water comes over (5.8 L was collected of butyl-vinyl-ether/water azeotrope). A solution of phosphoric acid (1.1 kg, 0.66 L, 6 Eq, 9.651 mol) and Acetylcysteine (131 g, 0.5 Eq, 804.2 mmol) in water (3.2 L) was prepared. The concentrated reaction mixture was poured into a 20 L reaction vessel. The flask was rinsed with (1.5 L) water and Toluene (1.5 L). Both rinses were added to the reaction vessel. The reaction mixture was cooled to 20° C. with an ice/water bath. The solution of phosphoric acid and acetyl cysteine was added to the reaction mixture slowly by a dropping funnel. A small exotherm to 25° C. and gas formation was observed. The temperature was kept below 25° C. The addition was complete after 1.5 hours. A brown suspension was obtained, which was stirred for 30 minutes.

The solids were collected by filtration over a 4 L P2 glass filter. The solids were washed 3 times with 2 L of toluene (each washing was kept separate). The filter cake was orange. The acidic water layer (dark brown/black) was washed successively with the toluene washing obtained after washing the filter cake.

To a 50 L separating funnel was added the acidic water layer and the filter cake. Additional compound 7 (35 g) and toluene (6 L) was added. The mixture was made basic (pH 9.5) by addition of 25% ammonia (1 to 1.5 L was used), and was stirred for 30 minutes. The layers easily separated but the water layer still contained solids and some tarry black material. The mixture was filtered over a pad of Celite (5 cm thick). The pad was then washed with toluene (2×2 L), resulting in the formation of solids on the Celite. These solids were dissolved with warm toluene (about 5 L at 60° C.) and filtered again. The combined organic layer was washed with water (4×2 L). The final water layer was pH 7-8. The organic layer (about 26 L) was concentrated to about 15 L at 50° C. on the rotavap. The mixture was transferred to a 20 L reaction vessel equipped with teflon coated metal stirring propeller, reflux condenser and temperature probe. Demineralized water (3 L) and Acetylcysteine (131 g, 0.5 Eq, 804.2 mmol) were added to the mixture, and stirred at 45° C. overnight. The mixture was transferred to a 50 L separating funnel, and additional toluene (8 L) was added to dissolve the remaining solids. 25% aqueous ammonia (160 mL) was added and stirred for 10 minutes. The layers were allowed to separate. The solids present in the water layer were dissolved by stirring with warm toluene (2×4 L), followed by extraction. The combined organic layers were again washed with water (4×2 L). The final washing was pH 7-8. The organic layer was dried over sodium sulfate and stored (total volume about 36 L). The combined organic layer was dried over sodium sulfate and filtered over a 4 L P2 glass filter. The mixture was concentrated to about 3 L on the large scale rotavap at 50° C. under reduced pressure. A thick suspension was obtained and cooled to 15° C. The solids were collected by filtering over a 4 L P2 glass filter. The solids were washed with cold toluene (1-2 L). The solids were dried in an open container at room temperature. The mother liquor was evaporated to dryness. This gave a dark brown sticky solid (114 g).

The crude product (617 g) was checked by NMR, which showed desired product 8 at high purity. Compound 8 (617.5 g) was dissolved in dichloromethane (8 L) and stirred in a 20 L reactor equipped with teflon coated metal stirring propeller, reflux condenser and temperature probe. To the reactor is added about 21 wt % SiliaMET DMT (131 g). The vessel was heated at 37° C. overnight. The reaction mixture was allowed to cool to room temperature. The mixture was then filtered over a pad of Celite (2 cm thick in a 4 L P2 glass filter). The solids on the filter were washed with dichloromethane (2 L). The combined filtrate was evaporated to dryness under reduced pressure at 45° C. This gave compound 8 (602 g) as an off-white solid. The Pd content was determined to be 48.8 ppm.

### Example 2—Preparation of Citrate Salt Crystals

2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one (500 mg) is prepared as summarized in Example 1, which is mixed with citric acid (232.1 mg). The mixture is dissolved in 16 mL acetone and is agitated overnight. The acetone was removed under vacuum the following day. The product was subjected to additional drying under vacuum at 40° C. for a period of two days.

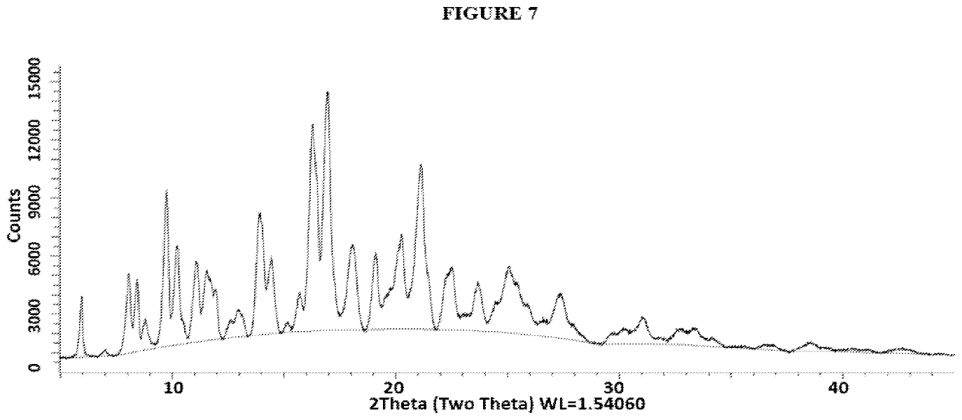

The XRPD of the citrate salt crystals is obtained as described or similarly described herein. The result is depicted in FIG. 7. The X-ray powder diffraction studies are performed using a Bruker AXS D8 discover HTS. Using a Cu anode at 40 kV, 40 mA; Göbel mirror, line optics. Detector: Linear detector LYNXEYE XE with receiving slit 2.950 detector opening. Measurement conditions: scan range 2-45° 2θ, 1 s/step, 0.005°/step, and all measuring conditions are logged in the instrument control file.

The XRPD pattern of the citrate Salt Crystals is depicted in FIG. 7 and has peaks as set forth below:

| Index | Angle | d Value | Relative Intensity |
|---|---|---|---|
| 1 | 5.899 | 14.96978 | 4.70% |
| 2 | 6.973 | 12.66677 | 100.00% |
| 3 | 7.796 | 11.3313 | 40.00% |
| 4 | 8.085 | 10.92637 | 4.00% |
| 5 | 8.766 | 10.07923 | 75.00% |
| 6 | 9.734 | 9.07874 | 2.80% |
| 7 | 10.088 | 8.76119 | 2.90% |
| 8 | 11.657 | 7.58558 | 32.90% |
| 9 | 11.931 | 7.41146 | 29.90% |
| 10 | 12.868 | 6.87432 | 3.40% |
| 11 | 13.156 | 6.72442 | 14.80% |
| 12 | 13.808 | 6.40803 | 19.70% |
| 13 | 13.97 | 6.33415 | 9.40% |
| 14 | 14.423 | 6.13647 | 28.60% |
| 15 | 15.061 | 5.87759 | 7.60% |
| 16 | 15.617 | 5.66965 | 22.30% |
| 17 | 16.147 | 5.48477 | 61.40% |
| 18 | 16.34 | 5.42028 | 12.50% |
| 19 | 16.812 | 5.26929 | 70.70% |
| 20 | 17.824 | 4.97236 | 8.90% |
| 21 | 18.085 | 4.90126 | 37.80% |
| 22 | 18.976 | 4.6729 | 32.00% |
| 23 | 19.305 | 4.59406 | 8.10% |
| 24 | 19.84 | 4.47137 | 10.70% |
| 25 | 20.187 | 4.3953 | 14.40% |
| 26 | 20.712 | 4.28513 | 13.30% |
| 27 | 21.011 | 4.2248 | 57.70% |
| 28 | 21.254 | 4.17707 | 12.50% |
| 29 | 22.065 | 4.02531 | 9.80% |
| 30 | 22.382 | 3.96901 | 13.40% |
| 31 | 23.619 | 3.76377 | 11.10% |
| 32 | 24.235 | 3.66947 | 3.80% |
| 33 | 24.949 | 3.5661 | 20.50% |
| 34 | 25.328 | 3.51362 | 11.00% |
| 35 | 26.484 | 3.36286 | 7.10% |
| 36 | 27.008 | 3.29876 | 9.80% |
| 37 | 27.222 | 3.27326 | 11.50% |
| 38 | 27.663 | 3.22208 | 4.10% |
| 39 | 29.532 | 3.02227 | 5.70% |
| 40 | 30.101 | 2.96647 | 4.70% |
| 41 | 30.83 | 2.89796 | 9.30% |
| 42 | 32.38 | 2.76265 | 4.60% |
| 43 | 38.313 | 2.34741 | 4.60% |

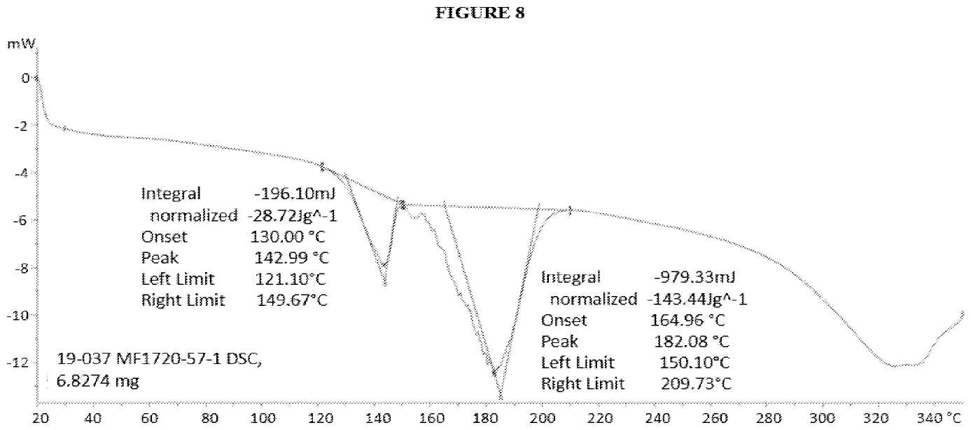

Differential Scanning Calorimetry (DSC) thermograph of the citrate Salt Crystals is obtained as described or similarly described herein and the DSC is depicted in FIG. 8. The DSC studies were performed using a Mettler Toledo DSC1 STARe System. The samples are made using Al crucibles (40 μl; pierced). 1-8 mg of sample is loaded onto a pre-weighed Al crucible and is kept at 20° C. for 5 minutes, after which it is heated at 10° C./min from 20° C. to 350° C. and kept at 350° C. for 1 minute. A nitrogen purge of 40 ml/min is maintained over the sample. The software used for data collection and evaluation is STARe Software v15.00 build 8668. No corrections are applied to the thermogram.

Thermogravimetric Analysis (TGA) of the citrate salt crystals is obtained as described or similarly described herein and is depicted in FIG. 6. The TGA/DSC studies were performed using a Mettler Toledo TGA/DSC-01/03 STARe System with a 34-position auto sampler. The samples are made using Al crucibles (40 μl; pierced). 5-10 mg of sample is loaded into a pre-weighed Al crucible and is kept at 20° C. for 5 minutes, after which it is heated at 10° C./min from 20° C. to 350° C. A nitrogen purge of 40 ml/min is maintained over the sample. The software used for data collection and evaluation is STARe Software v15.00 build 8668.

### Example 3—Preparation of Adipate Salt Crystals

2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one is prepared as summarized in Example 1, which is mixed with adipic acid. The mixture is dissolved in ethanol, acetone, or ethyl acetate at 50° C. The slurry was then cooled to a temperature of 20° C., and solids were removed.

The XRPD of the adipate salt crystals is obtained as described or similarly described herein. The result is depicted in FIG. 7. The X-ray powder diffraction studies are performed using a Bruker AXS D8 discover HTS. Using a Cu anode at 40 kV, 40 mA; Göbel mirror, line optics. Detector: Linear detector LYNXEYE XE with receiving slit 2.950 detector opening. Measurement conditions: scan range 2-45° 2θ, 1 s/step, 0.005°/step, and all measuring conditions are logged in the instrument control file.

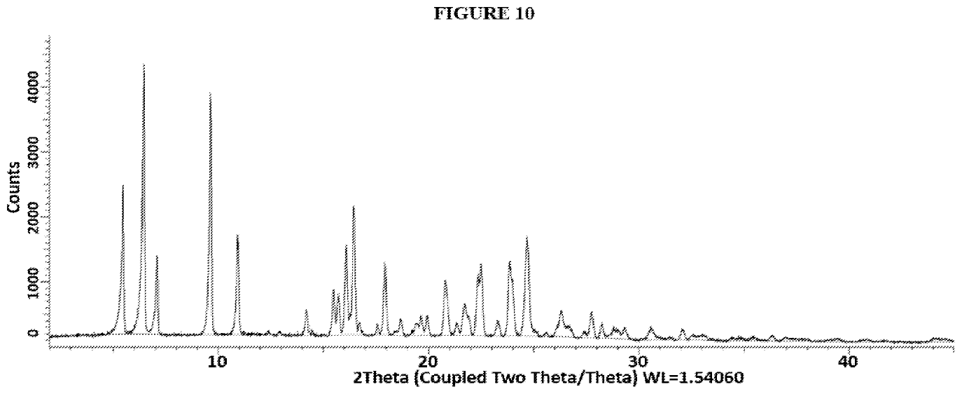

The XRPD pattern of the adipate Salt Crystals is depicted in FIG. 10 and has peaks as set forth below:

| Index | Angle | d Value | Relative Intensity |
|---|---|---|---|
| 1 | 5.439 | 16.23483 | 43.00% |
| 2 | 6.438 | 13.71747 | 95.60% |
| 3 | 7.07 | 12.49342 | 27.90% |
| 4 | 9.628 | 9.17907 | 100.00% |
| 5 | 10.918 | 8.09738 | 40.30% |
| 6 | 12.394 | 7.13586 | 1.40% |
| 7 | 12.923 | 6.84504 | 1.80% |
| 8 | 14.195 | 6.23423 | 11.40% |
| 9 | 15.525 | 5.70302 | 16.40% |
| 10 | 15.684 | 5.6455 | 13.90% |
| 11 | 16.098 | 5.50148 | 40.20% |
| 12 | 16.45 | 5.38442 | 57.00% |
| 13 | 16.712 | 5.30055 | 4.90% |
| 14 | 17.596 | 5.0362 | 4.70% |
| 15 | 17.937 | 4.94129 | 32.10% |
| 16 | 18.683 | 4.74569 | 6.60% |
| 17 | 19.498 | 4.54904 | 5.40% |
| 18 | 19.615 | 4.52223 | 5.90% |
| 19 | 19.939 | 4.4494 | 8.90% |
| 20 | 20.833 | 4.26041 | 23.20% |
| 21 | 21.362 | 4.1562 | 5.50% |
| 22 | 21.756 | 4.08176 | 12.40% |
| 23 | 22.443 | 3.95834 | 23.50% |
| 24 | 23.307 | 3.81343 | 5.90% |
| 25 | 23.913 | 3.71817 | 29.30% |
| 26 | 24.694 | 3.60233 | 44.10% |
| 27 | 25.6 | 3.47692 | 1.60% |
| 28 | 26.324 | 3.38294 | 11.20% |
| 29 | 26.609 | 3.34724 | 4.40% |
| 30 | 27.451 | 3.24647 | 2.60% |
| 31 | 27.761 | 3.21095 | 10.90% |
| 32 | 28.26 | 3.15538 | 5.50% |
| 33 | 28.899 | 3.08708 | 3.10% |
| 34 | 29.329 | 3.04273 | 5.30% |
| 35 | 30.595 | 2.91963 | 4.60% |
| 36 | 31.481 | 2.83947 | 1.20% |
| 37 | 32.091 | 2.78691 | 4.00% |
| 38 | 32.598 | 2.74467 | 2.00% |
| 39 | 33.046 | 2.70847 | 1.70% |
| 40 | 34.479 | 2.59915 | 1.50% |
| 41 | 34.818 | 2.57461 | 1.90% |
| 42 | 35.455 | 2.5298 | 1.00% |
| 43 | 36.353 | 2.46936 | 2.10% |
| 44 | 37.01 | 2.42703 | 1.50% |

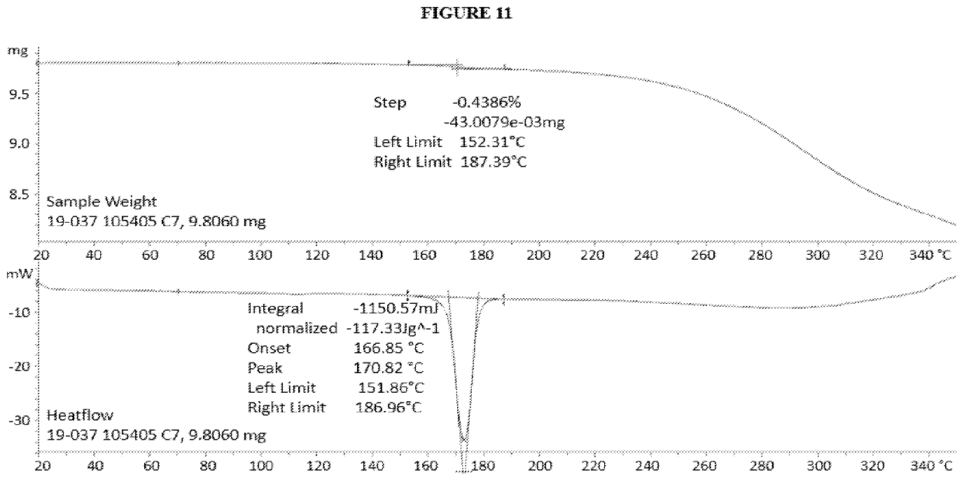

Thermogravimetric Analysis (TGA) & Differential Scanning Calorimetry (DSC) of the adipate salt crystals is obtained as described or similarly described herein and is depicted in FIG. 11. The TGA/DSC studies were performed using a Mettler Toledo TGA/DSC-01/03 STARe System with a 34-position auto sampler. The samples are made using Al crucibles (40 μl; pierced). 5-10 mg of sample is loaded into a pre-weighed Al crucible and is kept at 20° C. for 5 minutes, after which it is heated at 10° C./min from 20° C. to 350° C. A nitrogen purge of 40 ml/min is maintained over the sample. The software used for data collection and evaluation is STARe Software v15.00 build 8668.

### Example 4—Preparation of Malate Salt Crystals

2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one is prepared as summarized in Example 1, which is mixed with malic acid. The mixture is dissolved in acetonitrile at room temperature for 16 hours. The acetonitrile was pipetted off, and the remaining solvent was removed under vacuum. The product was subjected to additional drying under vacuum at room temperature for one day.

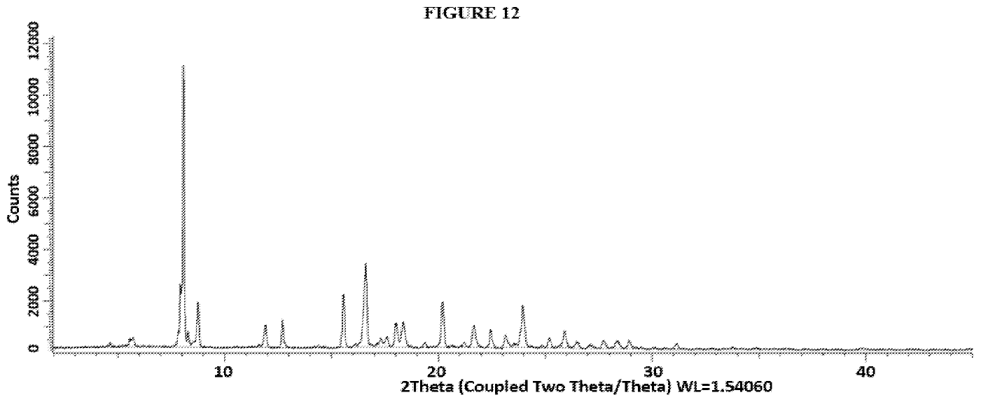

The XRPD of the malate salt crystals is obtained as described or similarly described herein. The result is depicted in FIG. 12. The X-ray powder diffraction studies are performed using a Bruker AXS D8 discover HTS. Using a Cu anode at 40 kV, 40 mA; Göbel mirror, line optics. Detector: Linear detector LYNXEYE XE with receiving slit 2.950 detector opening. Measurement conditions: scan range 2-45° 2θ, 1 s/step, 0.005°/step, and all measuring conditions are logged in the instrument control file.

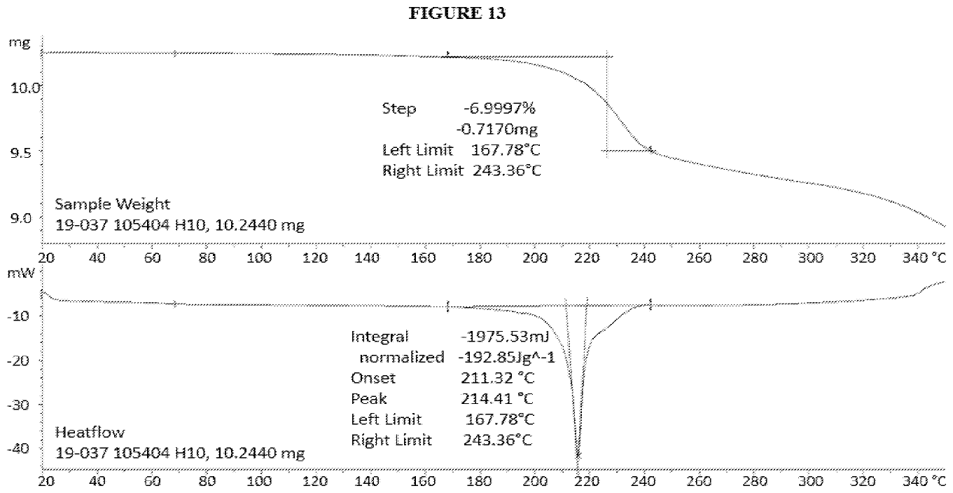

Thermogravimetric Analysis (TGA) & Differential Scanning Calorimetry (DSC) of the malate salt crystals is obtained as described or similarly described herein and is depicted in FIG. 13. The TGA/DSC studies were performed using a Mettler Toledo TGA/DSC-01/03 STARe System with a 34-position auto sampler. The samples are made using Al crucibles (40 μl; pierced). 5-10 mg of sample is loaded into a pre-weighed Al crucible and is kept at 20° C. for 5 minutes, after which it is heated at 10° C./min from 20° C. to 350° C. A nitrogen purge of 40 ml/min is maintained over the sample. The software used for data collection and evaluation is STARe Software v15.00 build 8668.

### Example 5—Preparation of Tartrate Salt Crystals

2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one is prepared as summarized in Example 1, which is mixed with tartaric acid. The mixture is dissolved in acetone or acetonitrile. at 50° C. The slurry was then cooled to a temperature of 20° C., and solids were removed.

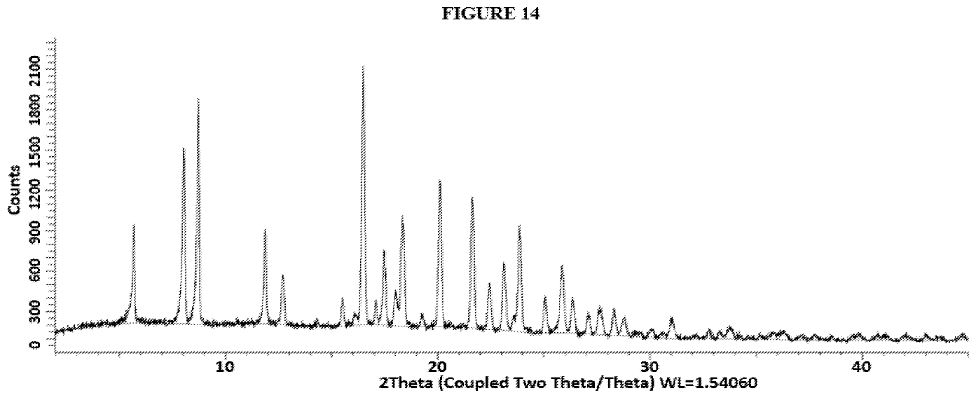

The XRPD of the tartrate salt crystals is obtained as described or similarly described herein. The result is depicted in FIG. 14. The X-ray powder diffraction studies are performed using a Bruker AXS D8 discover HTS. Using a Cu anode at 40 kV, 40 mA; Göbel mirror, line optics. Detector: Linear detector LYNXEYE XE with receiving slit 2.95° detector opening. Measurement conditions: scan range 2-45° 2θ, 1 s/step, 0.005°/step, and all measuring conditions are logged in the instrument control file.

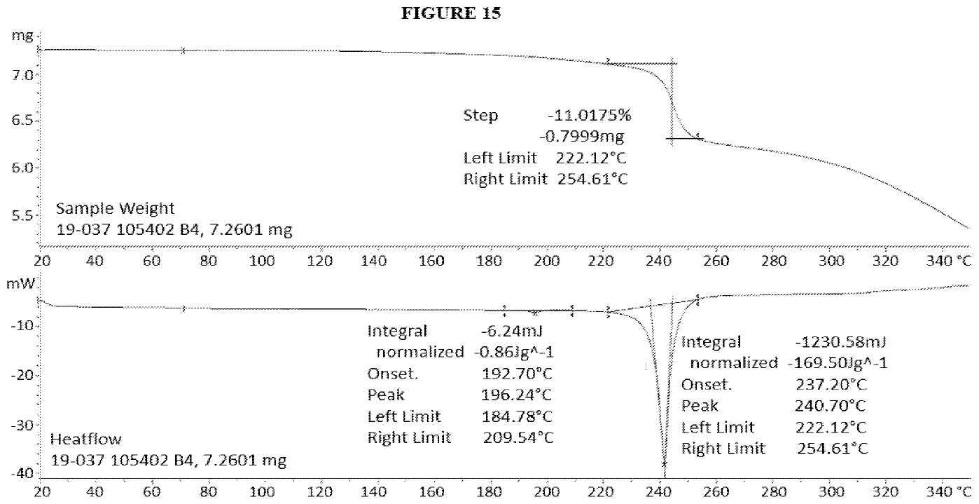

Thermogravimetric Analysis (TGA) & Differential Scanning Calorimetry (DSC) of the tartrate salt crystals is obtained as described or similarly described herein and is depicted in FIG. 15. The TGA/DSC studies were performed using a Mettler Toledo TGA/DSC-01/03 STARe System with a 34-position auto sampler. The samples are made using Al crucibles (40 μl; pierced). 5-10 mg of sample is loaded into a pre-weighed Al crucible and is kept at 20° C. for 5 minutes, after which it is heated at 10° C./min from 20° C. to 350° C. A nitrogen purge of 40 ml/min is maintained over the sample. The software used for data collection and evaluation is STARe Software v15.00 build 8668.

### Example 6—Preparation of Gluconate Salt Crystals

2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one is prepared as summarized in Example 1, which is mixed with gluconic acid. The mixture is dissolved in DMSO at room temperature for 16 hours. Excess DMSO was removed, and the product was subjected to additional drying under vacuum at room temperature for one day.

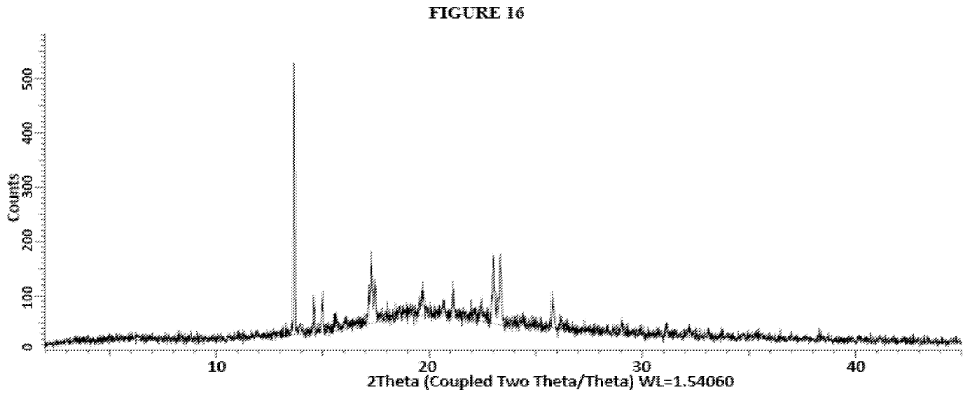

The XRPD of the gluconate salt crystals is obtained as described or similarly described herein. The result is depicted in FIG. 16. The X-ray powder diffraction studies are performed using a Bruker AXS D8 discover HTS. Using a Cu anode at 40 kV, 40 mA; Göbel mirror, line optics. Detector: Linear detector LYNXEYE XE with receiving slit 2.950 detector opening. Measurement conditions: scan range 2-45° 2θ, 1 s/step, 0.005°/step, and all measuring conditions are logged in the instrument control file.

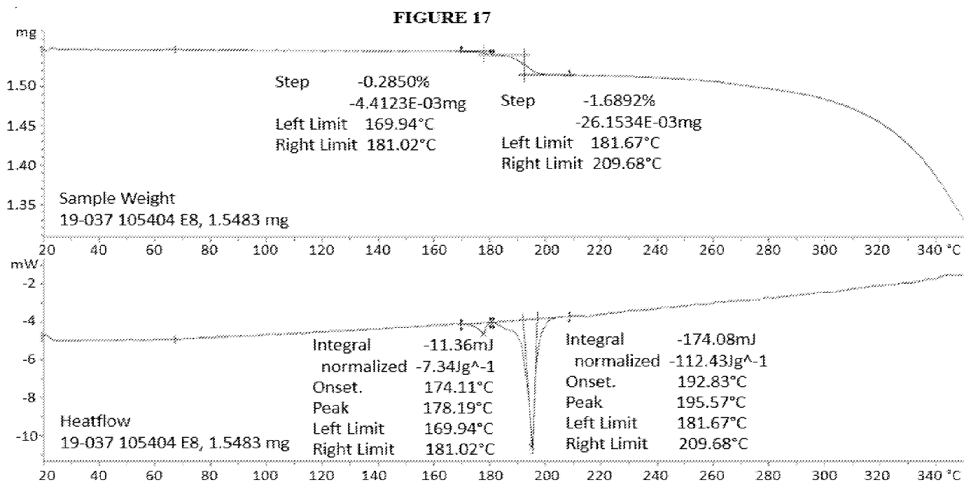

Thermogravimetric Analysis (TGA) & Differential Scanning Calorimetry (DSC) of the gluconate salt crystals is obtained as described or similarly described herein and is depicted in FIG. 17. The TGA/DSC studies were performed using a Mettler Toledo TGA/DSC-01/03 STARe System with a 34-position auto sampler. The samples are made using Al crucibles (40 μl; pierced). 5-10 mg of sample is loaded into a pre-weighed Al crucible and is kept at 20° C. for 5 minutes, after which it is heated at 10° C./min from 20° C. to 350° C. A nitrogen purge of 40 ml/min is maintained over the sample. The software used for data collection and evaluation is STARe Software v15.00 build 8668.

### Example 7—Solubility Study of Succinate Salt Crystals

The solubilities of free base crystalline and succinate salt crystalline forms of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one are compared. Samples are titrated in water or in a minimum of three titrations under co-solvent conditions from the pH where the sample is fully dissolved. The sample precipitates from solution are detected by a UV-turbidity probe, which corresponds to a kinetic solubility. After precipitation, base and acid titrants are alternately added to drive the sample back and forth across the equilibrium solubility of the neutral species (the intrinsic solubility). At this point, the samples would exist in a supersaturated or subsaturated state (i.e. chase equilibrium). The intrinsic solubilities are determined from the pH between the supersaturated and subsaturated states corresponding to an intrinsic solubility. The samples can be determined by extrapolation to aqueous media, when co-solvent conditions are used.

The solubility of succinate salt is about 7 mg/mL, significantly higher than free base (0.285 mg/mL). This degree of aqueous solubility predicts faster dissolution rates in vitro and in vivo.

### Example 8—Pharmacokinetics Study of Succinate Salt Crystals in Dogs

The succinate salt of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one is administered to dogs at a dose of 5 mg/kg orally. A separate group of dogs is administered 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one free base 5 mg/kg orally. The analysis of drug concentration in plasma samples collected is analyzed.

The pharmacokinetic (PK) parameters are determined from the plasma concentration versus time data by non-compartmental methods with uniform weighting. The maximum observed concentration (Cₘₐₓ) and the time of the maximum observed concentration (Tₘₐₓ) are obtained from the bioanalytical raw data. The area-under-the-plasma concentration-time curve from time zero to the time of the last measurable sample (AUC) is calculated by the trapezoidal rule. The plasma pharmacokinetic profile of the free base and the succinate salt crystal in 5 mg/kg dosage is provided in Table 5 below.

TABLE 5

| | Succinate Salt | Free Base |
|---|---|---|
| Cₘₐₓ (ng/mL) | 619.0 | 204.8 |
| Tₘₐₓ (hr) | 0.4 | 1.3 |
| T_{1/2} (hr) | 7.0 | 6.8 |
| AUC₀₋₂₄ (ng · hr/mL) | 1659.7 | 1523 |
| DN Cₘₐₓ | 123.8 | 41 |
| ([ng/mL]/[mg/kg]) | | |
| DN AUC₀₋₂₄ | 331.9 | 304.6 |
| ([ng · hr/mL]/[mg/kg]) | | |

1. A crystal of the compound 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one in free base form.

2. The crystal according to claim 1, wherein the crystal is in non-solvate form.

3. The crystal according to claim 1, wherein the free base crystal is in solvate form with methanol, ethanol, propanol or butanol.

4. The crystal according to claim 1, wherein the crystal exhibits an X-ray powder diffraction pattern comprising at least five peaks having 2-theta angle values selected from the group consisting of: 9.3, 14.0, 14.7, 17.3, 17.9, 18.7, 21.2, 23.2, 23.3, and 23.7 degrees, wherein the XRPD pattern is measured in a diffractometer using copper anode at wavelength alpha1 of 1.5406 Å and wavelength alpha2 of 1.5444 Å.

5. The crystal according to claim 1, wherein the crystal exhibit an X-ray powder diffraction pattern comprising at least five peaks having d-spacing values selected from the group consisting of 9.53, 6.33, 6.02, 5.11, 4.95, 4.74, 4.19, 3.83, 3.82, 3.79 Å.

6. The crystal according to claim 1, wherein said free base crystal exhibits a Differential Scanning Calorimetry (DSC) pattern comprising an endothermic peak at about 195° C.-196° C.

7. A crystal of the compound 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one in acid addition salt form.

8. The crystal according to claim 7, wherein the salt is a succinate salt.

9. The crystal according claim 8, wherein the salt is a succinate salt having a free base to succinic acid molar ratio of 1:1 or 2:1.

10. The crystal according to claim 8, wherein the salt is a mono-succinate salt.

11. The crystal according to claim 8, wherein the Salt Crystals exhibit an X-ray powder diffraction pattern comprising at least five peaks having 2-theta angle values selected from the group consisting of: 7.8, 8.2, 11.6, 14.5, 16.5, 18.6, 19.7, 20.4, 20.6, 22.1, 23.3, 24.8, 26.0, and 28.5 degrees, wherein the XRPD pattern is measured in a diffractometer using copper anode, e.g., at wavelength alpha1 of 1.5406 Å and wavelength alpha2 of 1.5444 Å.

12. The crystal according to claim 8, wherein said salt crystals exhibit an X-ray powder diffraction pattern comprising at least five peaks having d-spacing values selected from the group consisting of 11.37, 10.77, 7.62, 6.09, 5.38, 4.77, 4.50, 4.36, 4.31, 4.02, 3.81, 3.59, 3.43, and 3.13 Å.

13. The crystal according to claim 8, wherein said salt crystal exhibits a Differential Scanning Calorimetry (DSC) pattern comprising an endothermic peak at about 177° C.-178° C.

14-37. (canceled)

38. A method for the production of acid addition salts of 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one (“Compound A”) comprising the steps of reacting Compound A with an acid in a solvent and isolating the salt obtained.

39. The method according to claim 38, wherein the acid is selected from citric acid, adipic acid, tartaric acid, malic acid, succinic acid, gluconic acid, maleic acid, fumaric acid, aspartic acid, hippuric acid, sebacic acid, glycolic acid, galactaric acid, benzoic acid, pamoic acid, oxalic acid and malonic acid.

40. The method according to claim 38, wherein the solvent is an alcohol, acetone, acetonitrile, dimethyl sulfoxide (DMSO), ethyl acetate, and/or toluene.

41. The method according to claim 38, wherein 2-(4-acetylbenzyl)-3-((4-fluorophenyl)amino)-5,7,7-trimethyl-7,8-dihydro-2H-imidazo[1,2-a]pyrazolo[4,3-e]pyrimidin-4(5H)-one (Compound A) is in crystalline form.

42. The crystal according to claim 7, wherein the acid addition salt form of Compound A is selected from the group consisting of citrate, adipate, tartrate, malate, gluconate, maleate, fumarate, aspartate, hippurate, sebacate, glycolate, galactarate, benzoate, pamoate, oxalate and malonate salt form.
